(19) United States
(12) Patent Application Publication
(10) Pub. No.: US 2004/0259947 A1
(43) Pub. Date: Dec. 23, 2004

(54) Glycerin fatty acid ester for palliate a symptom of premenstrual syndrome, a palliative of premenstrual syndrome, oil or fat composition for palliate a symptom of premenstrual syndrome, and food and drink for palliate a symptom of premenstrual syndrome

(76) Inventors: Shinji Seki, Yokohama-shi (JP); Yuuko Hirasawa, Yokohama-shi (JP); Miho Sakurada, Miura-shi (JP); Yuri Arai, Yokohama-shi (JP); Seiichi Shirasawa, Yokohama-shi (JP); Satoshi Negishi, Tokyo (JP)

Correspondence Address: MICHAELSON AND WALLACE, PARKWAY 109 OFFICE CENTER, 328 NEWMAN SPRINGS RD, P O BOX 8489, RED BANK, NJ, 07701

(21) Appl. No.: 10/875,080

(22) Filed: Jun. 23, 2004

Related U.S. Application Data
(63) Continuation-in-part of application No. 09/953,852, filed on Sep. 17, 2001, now abandoned.

(51) Int. Cl. A61K 31/225; A61K 35/78

(52) U.S. Cl. 514/547; 554/176; 424/776

(57) ABSTRACT

It is an object of the present invention to provide a glycerin fatty acid ester having an effect of mitigating the symptoms associated with the premenstrual syndromes (hereunder referred to as PMS) and excellent in the immediate effect as well as a drug for mitigating the symptoms associated with the PMS, a food or drink for mitigating the symptoms associated with the PMS, an edible fats and oils-containing composition for mitigating the symptoms associated with the PMS or the like, which comprise the glycerin fatty acid ester.

### RELATED CO-PENDING PATENT APPLCIATION

This application claims the benefit of our co-pending United States patent application entitled “Glycerin Fatty Acid Ester for Palliate a Symptom of Premenstrual Syndrome, a Palliative of Premenstrual Syndrome, Oil or Fat Composition for Palliate a Symptom of Premenstrual Syndrome, and Food and Drink for Palliate a Symptom of Premenstrual Syndrome” filed Sep. 17, 2001 and assigned Ser. No. 09/953,852, which is incorporated by reference herein.

### FIELD OF THE INVENTION

Glycerin fatty acid ester for palliate a symptom of Premenstrual Syndrome, a palliative of Premenstrual Syndrome, oil or fat composition for palliate a symptom of Premenstrual Syndrome, and food and drink for palliate a symptom of Premenstrual Syndrome

### SUMMARY OF THE INVENTION

The present invention relates to a glycerin fatty acid ester used for mitigating or relieving the symptoms associated with premenstrual syndromes (hereunder simply referred to as “PMS”), a drug, a food or drink and an edible fats and oils-containing composition for mitigating the symptoms associated with PMS comprising the glycerin fatty acid ester. More specifically, the present invention pertains to a glycerin fatty acid ester used for mitigating or relieving the symptoms associated with the PMS, which are highly absorbable and excellent in the immediate effect.

### BACKGROUND OF THE INVENTION

The term “PMS” means the physical symptoms or psychic (or mental) symptoms caused about one week before the menstruation (=menses) (corpus luteum-activated phase), which was first reported by Frank in 1931 and it is defined, on the medical standpoint, as follows: “The PMS means physical and psychic symptoms initiated before 3 to 10 days from the initiation of the menstruation and attenuated or disappeared in response to the initiation of the menstruation” (The Society of Obstetrics and Gynecology in Japan). The term “PMS” is also referred to as “Premenstrual Tension”. In addition, there have been proposed various expressions or terminology such as “premenstrual experience”, for the term “PMS” in order to release women from the negative image derived from the medical terms such as premenstrual syndromes and premenstrual tension, to prevent them from falling a prey to the medical institution, to change the negative impression of these terms to more positive impression and to thus care the symptoms by themselves.

The representative symptoms derived from the PMS include, for instance, such physical symptoms as abdominal pains, abdominal inflation, lumbago, mastalgia, stiffness in the shoulders, headache, promotion of appetite, roughened skin, acne, diarrhea and constipation; and such psychic symptoms as irritation, melancholy, touchiness, feeling tired of doing anything, use of strong languages to others and a desire to rearrange something and put them in order.

There are observed a large number of women prior to the menopause, who suffer from such PMS or symptoms during the menstruation and it would be assumed that more than half of women prior to the menopause may suffer from some symptoms although the degree of seriousness thereof may vary case by case. Moreover, such symptoms would interfere with the daily life in case of a serious patient and these symptoms become a cause of worsening of the personal relations of the patient with her jobsite, her family and her friends and even a cause of committing a crime. Moreover, there is a scholar who has pointed out the correlation between the PMS syndromes and the productivity of women's labor, under such a condition that the women have increasingly participated in the public affairs.

Under the circumstances, it would be an important social problem to relieve and/or mitigate these symptoms and to improve the women's QOL (Quality of Life).

To solve such a problem, there have been conducted various researches from the standpoint of, for instance, the medical science, the science of nutrition and the science of nursing. For instance, a method has attracted interest recently from the standpoint of the science of nutrition, which comprises administering fats and oils containing an essential fatty acid as a precursor of prostaglandin to a patient. More specifically, there have been reported papers or articles concerning the mitigation and alleviation of the PMS symptoms by the intake of a fats and oils-containing composition, which comprises γ-linolenic acid as a precursor, and/or a fats and oils-containing composition comprising di-homo-γ-linolenic acid. In addition, these articles have been opened to the public as prior arts.

However, these methods do not often ensure sufficient effects in the alleviation and mitigation of the PMS symptoms superior to the placebo effects and further the compositions used in these methods are still insufficient in the immediate effect, the ability of being absorbed (absorbability), the ability of preventing the body fat-accumulation, the taste and the stability in quality.

In particular, the patient would be reluctant to regularly and continuously ingest such a composition because of the possibility of the body fat-accumulation due to the intake of the fats and oils and the regular and continuous intake thereof would be quite tedious for the patient. Accordingly, there has been desired for the development of a product ensuring, in particular, an immediate effect.

Moreover, if using such a composition as a general food (or a health food (an enriched food)), it is necessary to ingest a relatively large amount of fatty acids over a long period of time to ensure a sufficient desired effect. For this reason, the relatively younger women prior to the menopause ranging from 20-year-old to 40-year-old as the subjects for these treatments would entertain some fear for the possibility of the body fat-accumulation and they would also suffer economic and physical burdens.

Moreover, the fats and oils-composition suffers from a problem of the stability in the quality thereof due to the incorporation of highly unsaturated fatty acids therein and for this reason, there is a limit in the expansion of the applications of the composition to the general foods. This would, in turn, result in such a practical problem that women lose their opportunity of ingesting the composition. Further, the fatty acid has a flavor peculiar thereto and such a flavor is not commonly acceptable.

More specifically, articles such as those reported by Casper et al (1987), Khoo et al (1990) and Collins et al (1993) disclose the results obtained by the tests for evaluating the alleviation effect of oil derived from evening primrose oil containing γ-linolenic acid in the form of triglyceride on the PMS symptoms, the tests being carried out according to the double blind crossover test. The article of Collins et al reported that panelists ingested 6 g/day of the oil or 480 mg/day of γ-linolenic acid over a long period of time on the order of 8 weeks, but any significant effect was not observed at all, when comparing with the effect observed for the control group (who ingested liquid paraffin).

In addition, Japanese Un-Examined Patent Publication (hereunder simply referred to as “J. P. KOKAI”) No. Sho 54-117035 discloses “A pharmaceutical composition for mitigating the PMS symptoms, which comprises γ-linolenic acid or a physiologically functional derivative thereof and/or di-homo-γ-linolenic acid or a physiologically functional derivative thereof or a combination thereof with a pharmaceutically acceptable vehicle”.

However, this patent simply discloses the application of γ-linolenic acid and/or di-homo-γ-linolenic acid or physiologically functional derivatives thereof to the treatment of the emmeniopathy, never points out such problems as the immediate effect, the ability of being absorbed, the flavor and the stability in quality of the composition in the alleviation of the symptoms associated with the premenstrual syndromes and never discloses any solution of these problems.

Moreover, J. P. KOKAI No. Sho 62-16415 also discloses a pharmaceutical composition for treating the PMS symptoms, which comprises at least one member selected from the group consisting of γ-linolenic acid, di-homo-γ-linolenic acid, arachidonic acid, 22:4n-6 and 22:5n-6, which are metabolites of linolenic acid as an essential fatty acid; and at least one member selected from the group consisting of α-linolenic acid and 18:4n-3, 20:4n-3, 20:5n-3, 22:5n-3 and 22:6n-3, which are metabolites of α-linolenic acid, in which these substances may be used as it is, or in the form of esters, salts, amides or other derivatives capable of being converted into acids in the living body, and which are used alone or in any combination with pharmaceutically acceptable carriers or diluents. However, this patent never points out the foregoing problems and does not disclose any solution thereof, just like J. P. KOKAI No. Sho 54-117035.

Furthermore, J. P. KOKAI Nos. Sho 63-77817 and Hei 5-201924 disclose a γ-linolenic acid-containing fats and oils-composition for mitigating the PMS symptoms.

Among these patent applications, J. P. KOKAI No. Sho 63-77817 suggests that the simultaneous use of γ-linolenic acid and a calcium salt may exert influence on the effect of γ-linolenic acid. However, this patent application does not refer to the influence of the structures of the fats and oils used, on the effect of alleviating the PMS symptoms.

On the other hand, it would be assumed that J. P. KOKAI No. Hei 5-201924 refers to the influence of the difference in the fats and oils structure on the alleviation effect, but this article, as a rule, simply refers to the concentration and effect of di-linoleoyl-mono-γ-linolenyl-glycerol (DLMG), which is a component constituting triglycerides contained in evening primrose oil fats and oils and borage oil, used as supplementary raw materials for naturally occurring γ-linolenic acid.

As has been discussed above in detail, none of the foregoing prior arts discloses that the fats and oils-containing composition comprises γ-linolenic acid as a fatty acid moiety constituting triglycerides, that the rate of the middle chain fatty acids with respect to all of the fatty acids present in the composition exert influence on the body fat-accumulating ability and ability of being absorbed by the living body and that the rate of the 1- and/or 3-γ-linolenic acid binding sites on a triglyceride exerts influence on, for instance, the immediate effect of alleviating the symptoms, the quality stability and the flavor of the resulting composition. Further, none of these prior arts refers to the fact that the compositions are excellent in these points as compared with the conventional γ-linolenic acid-containing fats and oils.

In addition, J. P. KOKAI No. Hei 4-501812 discloses that low calorie fats and oils can be provided through the use of triglycerides formed from long chain fatty acids and short chain fatty acids. However, the triglyceride consisting of short chain fatty acids gives out a smell peculiar thereto. Therefore, the fats and oils are limited in the cooked products to which the fats and oils can be applied and the fats and oils-containing product is unfavorable as a widely used edible oil product. Moreover, it has been known that the middle chain fatty acids are easily converted into an energy and therefore, they have a low body fat-accumulating ability (J. Lipid Res., 1996, 37:708-726). In this respect, the triglycerides formed from middle chain fatty acids are, by nature, highly safe, but there have been reported that they cause symptoms such as diarrhea, naused feeling, stomachache, pyrosia and anorexia, if one ingests a large amount of the same at a time.

J. P. KOKAI Nos. Hei 4-300826, Hei 8-60180 and Hei 10-176181 disclose a fats and oils-containing composition containing a diglyceride as an effective component, which has a low ability of accumulating body fat. However, there has not yet been completely elucidated the safety of the fats and oils-containing composition having a high content of the diglyceride when one ingests the same over a long period of time. Moreover, it is presently difficult to economically produce diglyceride at a high concentration and therefore, it is difficult to widely use diglycerides from the economical standpoint.

Moreover, J. P. KOKAI No. Hei 8-269478 discloses a fats and oils-containing composition whose ability of accumulating body fat is low and which comprises diglycerides and triglycerides, wherein it comprises not less than 31% by mass, based on the total mass of the composition, of a triglyceride having two middle chain fatty acid residues in the molecule. The invention disclosed therein likewise employs diglycerides as effective components and therefore, it suffers from the same problems as those pointed out above in connection with J. P. KOKAI Nos. Hei 4-300826, Hei 8-60180 and Hei 10-176181.

In addition, Japanese Examined Patent Publication (hereunder simply referred to as “J. P. KOKOKU”) No. Hei 4-4358 discloses a composition comprising fats and oils, which contain triglycerides consisting of middle fatty acids and γ-linolenic acid. However, J. P. KOKOKU No. Hei 4-4358 discloses that the composition is used for treating patients suffering from, for instance, disorders in energy supply (such as resuscitation and dystrophic conditions) and metabolic deficiencies such as disorders in lipid-digestion or diabetes and never discloses the effect of the present invention or the alleviation of the symptoms associated with PMS.

### DETAILED DESCRIPTION OF THE INVENTION

Accordingly, it is an object of the present invention to provide a glycerin fatty acid ester for mitigating the symptoms associated with PMS, a drug for mitigating the symptoms associated with PMS, a food or drink for mitigating the symptoms associated with PMS and a fats and oils-containing composition for mitigating the symptoms associated with PMS, which are used for mitigating the PMS symptoms, which have a symptom-alleviation effect, in particular, an excellent immediate effect of mitigating the PMS symptoms and which are further excellent in, for instance, the body fat-accumulation-inhibitory effect and have high absorbability of γ-linolenic acid and good taste and texture as well as high quality stability.

Means for Solving the Problems

The inventors of this invention have conducted various studies to achieve the foregoing object and have found that a glycerin fatty acid ester carrying a γ-linolenic acid residue at the 1- or 3-position thereof has an effect of mitigating the symptoms associated with PMS and possesses an excellent immediate effect. Simultaneously, the inventors have also found that the foregoing effect is further improved by blending fats and oils containing a short chain or middle chain fatty acid with fats and oils carrying γ-linolenic acid residues and then subjecting the resulting blend to transesterification.

Further, the rate of γ-linolenic acid residues linked at 1-, 3-sites as a fatty acid moieties constituting the fats and oils and the rate of the short chain or middle chain fatty acid residues are closely correlated with the body fat-accumulating ability, the absorbability, the immediate effect of mitigating the symptoms, the quality stability and the taste and texture of the resulting composition. Thus, the inventors of this invention have completed the present invention.

Accordingly, the present invention provides a glycerin fatty acid ester for mitigating the symptoms associated with premenstrual syndromes, represented by the following general formula (I):

R₁O—CH₂—CH(OR₂)—CH₂—OR₃ (I)

wherein R₁, R₂ and R₃ each represents a hydrogen atom or a fatty acid residue having 2 to 24 carbon atoms, provided that at least one of R₁ and R₃ represents a γ-linolenic acid residue.

According to another aspect of the present invention, there is also provided a drug for mitigating the symptoms associated with PMS, which comprises, as an effective component, the foregoing glycerin fatty acid ester. The foregoing glycerin fatty acid ester and the drug for mitigating the symptoms associated with PMS possess an immediate effect of mitigating the symptoms associated with PMS.

According to a further aspect of the present invention, there is provided a drug for mitigating the symptoms associated with premenstrual syndromes further comprises at least one member selected from the group consisting of vitamin B's, vitamin E, herbs and mineral salts, in addition to the foregoing glycerin fatty acid ester.

According to the present invention, there are further provided the foregoing drug for mitigating the symptoms associated with premenstrual syndromes, wherein the herb is at least one member selected from the group consisting of ginkgo leaves, passionflower, lemon burm and ginger; the foregoing drug for mitigating the symptoms associated with premenstrual syndromes, wherein the herb is at least one member selected from the group consisting of ginkgo leaves, passionflower and ginger and it can mitigate the symptoms caused by hemokinesis-inhibition; the foregoing drug for mitigating the symptoms associated with premenstrual syndromes, wherein the herb is at least one member selected from the group consisting of ginkgo leaves, passionflower and ginger and it can mitigate the positive psychic symptoms; the foregoing drug for mitigating the symptoms associated with premenstrual syndromes, wherein the herb is at least one member selected from the group consisting of chamomile and lemon burm and it can mitigate the symptoms of digestive system; the foregoing drug for mitigating the symptoms associated with premenstrual syndromes, wherein the herb is at least one member selected from the group consisting of chamomile and lemon burm and it can mitigate the symptoms associated with negative psychic syndromes; the foregoing drug for mitigating the symptoms associated with premenstrual syndromes, wherein the herb is at least one member selected from the group consisting of feverfew and rosemary and it can mitigate dolorific symptoms; the foregoing drug for mitigating the symptoms associated with premenstrual syndromes, wherein the herb is at least one member selected from the group consisting of feverfew and rosemary and it can mitigate the easy susceptibility to fatigue; the foregoing drug for mitigating the symptoms associated with premenstrual syndromes, wherein the herb is at least one member selected from the group consisting of common dandelion and celery and it can mitigate the dropsy; the foregoing drug for mitigating the symptoms associated with premenstrual syndromes, wherein the herb is at least one member selected from the group consisting of common dandelion and celery and it can mitigate the reduction of the ability to concentration; and the foregoing drug for mitigating the symptoms associated with premenstrual syndromes, wherein the herb is at least one member selected from the group consisting of common dandelion and celery and it can mitigate the symptoms of mastalgia.

The present invention also provides a food or drink for mitigating the symptoms associated with premenstrual syndromes comprising the foregoing glycerin fatty acid ester for mitigating the symptoms associated with premenstrual syndromes. In addition, the present invention further provides a food or drink for mitigating the symptoms associated with premenstrual syndromes comprising the foregoing drug for mitigating the symptoms associated with premenstrual syndromes.

The present invention further provides an edible fats and oils-containing composition for mitigating the symptoms associated with premenstrual syndromes, which comprises the glycerin fatty acid ester for mitigating the symptoms associated with premenstrual syndromes, in which the total amount of the γ-linolenic acid residues present in R1 and those present in R3 is not less than 1% by mass on the basis of the total amount of the fatty acid residues present in the composition.

The present invention also provides the foregoing edible fats and oils-containing composition for mitigating the symptoms associated with premenstrual syndromes, wherein the ratio of the total amount of the γ-linolenic acid residues present in R1 and those present in R3 to the amount of the γ-linolenic acid residues present in R2 ranges from 1:0.1 to 1:3.

The present invention likewise provides the foregoing edible fats and oils-containing composition for mitigating the symptoms associated with premenstrual syndromes, wherein the total amount of the γ-linolenic acid residues present in R1, R2 and R3 is not less than 2% by mass on the basis of the total amount of the fatty acid residues present in the composition.

The present invention likewise provides the foregoing edible fats and oils-containing composition for mitigating the symptoms associated with premenstrual syndromes, wherein the total amount of the fatty acid residues having 2 to 12 carbon atoms present in R1, R2 and R3 is not less than 5% by mass on the basis of the total amount of the fatty acid residues present in the composition.

The present invention also provides an edible fats and oils-containing composition for mitigating the symptoms associated with premenstrual syndromes comprising the foregoing glycerin fatty acid ester for mitigating the symptoms associated with premenstrual syndromes, wherein the total amount of the γ-linolenic acid residues present in R₁, R2 and R3 is not less than 5% by mass on the basis of the total amount of the fatty acid residues present in the composition; the total amount of the fatty acid residues having 2 to 12 carbon atoms present in R1, R2 and R3 is not less than 10% by mass on the basis of the total amount of the fatty acid residues present in the composition; and 40 to 90% by mass of the total amount of the γ-linolenic acid residues present in R1, R2 and R3 is present in R1 or R3.

Moreover, the present invention also provides the foregoing edible fats and oils-containing composition for mitigating the symptoms associated with premenstrual syndromes, wherein it is prepared by transesterirying.

The present invention further provides the foregoing edible fats and oils containing composition for mitigating the symptoms associated with premenstrual syndromes wherein the composition is prepared by transesterifying fats and oils containing 5 to 95% by mass of γ-linolenic acid residues on the basis of the total amount of the fatty acid residues present in the composition.

Moreover, the present invention further provides the foregoing edible fats and oils-containing composition for mitigating the symptoms associated with premenstrual syndromes wherein the composition is prepared by transesterifying fats and oils containing 5 to 95% by mass of γ-linolenic acid residues and 5 to 40% by mass of fatty acid residues having 2 to 12 carbon atoms, on the basis of the total amount of the fatty acid residues present in the composition.

In addition, the present invention likewise provides the foregoing edible fats and oils-composition for mitigating the symptoms associated with premenstrual syndromes as set forth in claim 21, wherein the following substances (A) and/or (B) are prepared for use as a starting material and then subjected the substances (A) and/or (B) to transesterification:

(A): At least one member selected from the group consisting of borage oil, evening primrose oil and microorganism-fermented fats and oils; and

(B): At least one member selected from the group consisting of fatty acids each having 2 to 12 carbon atoms, glycerin fatty acid esters carrying 1 to 3 fatty acid residues having 2 to 12 carbon atoms and fats and oils containing the same.

The present invention also provide an edible fats and oils-containing composition for mitigating the symptoms associated with premenstrual syndromes, wherein it is prepared by transesterirying at ordinary pressure in a nitrogen gas stream or a reduced pressure of not more than 10 Pa at a temperature ranging from 80 to 120° C. for 10 to 60 minutes in the presence of sodium methylate as a catalyst.

In addition, the present invention also provides the foregoing edible fats and oils-containing composition for mitigating the symptoms associated with premenstrual syndromes as set forth in any one of claims 19 to 22 wherein it is prepared by transesterirying at a temperature ranging from 40 to 100° C. for 2 to 48 hours using an enzyme for decomposing lipids (a lipase).

The present invention further provides the foregoing edible fats and oils-containing composition for mitigating the symptoms associated with premenstrual syndromes wherein the rate of the triglycerides containing, in the molecule, 3 fatty acid residues having 2 to 12 carbon atoms is not more than 3% by mass on the basis of the total amount of the triglycerides present in the edible composition.

Moreover, the present invention provides a drug for mitigating the symptoms associated with premenstrual syndromes comprising the foregoing edible fats and oils-containing composition for mitigating the symptoms associated with premenstrual syndromes.

In other words, the present invention provides a drug and a food or drink for mitigating the symptoms associated with premenstrual syndromes each comprising an edible fats and oils-containing composition for mitigating the symptoms associated with premenstrual syndromes.

According to a further aspect of the present invention, there is provided an edible fats and oils-containing composition for mitigating the symptoms associated with premenstrual syndromes, characterized in that it comprises a glycerin fatty acid ester for mitigating the symptoms associated with premenstrual syndromes, wherein the following substances (A) and (B) are prepared for use as a starting material and then subjected the substances (A) and (B) to transesterification, and represented by the following general formula (I):

(A): At least one member selected from the group consisting of borage oil, oils derived from evening primrose and microorganism-fermented fats and oils; and

(B): At least one member selected from the group consisting of fatty acids each having 2 to 12 carbon atoms, glycerin fatty acid esters carrying 1 to 3 fatty acid residues having 2 to 12 carbon atoms and fats and oils containing the same,

R₁O—CH₂—CH(OR₂)—CH₂—OR₃ (I)

wherein R₁, R₂ and R₃ each represents a hydrogen atom or a fatty acid residue having 2 to 24 carbon atoms, provided that at least one of R₁ and R₃ represents a γ-linolenic acid residue, at least one of R₁, R₂ and R₃ represents a fatty acid residue having 2 to 12 carbon atoms, and the total amount of the γ-linolenic acid residues present in R₁ and those present in R₃ is not less than 1% by mass on the basis of the total amount of the fatty acid residues present in the composition, and the ratio of the total amount of the γ-linolenic acid residues present in R1 and those present in R3 to that of γ-linolenic acid residues present in R₂ ranges from 1:0.1 to 1:3.

Best Mode for Carrying Out the Invention

The inventors of this invention have found that the rate of the γ-linolenic acid residues linked to the 1- and/or 3-sites of a triglyceride as constituent fatty acid moieties and the rate of the short chain or middle chain fatty acid residues are closely correlated with the body fat-accumulating ability, the absorbability, the immediate effect of mitigating the symptoms associated with premenstrual syndromes, the quality stability and the flavor of the composition and thus have completed the present invention.

The present invention will hereunder be described in more detail.

The present invention relates to a glycerin fatty acid ester for mitigating the symptoms associated with premenstrual syndromes, represented by the following general formula (I):

R₁O—CH₂—CH(OR₂)—CH₂—OR₃ (I)

wherein R₁, R₂ and R₃ each represents a hydrogen atom or a fatty acid residue having 2 to 24 carbon atoms, provided that at least one of R₁ and R₃ represents a γ-linolenic acid residue.

In many cases, conventionally proposed drugs for mitigating the symptoms associated with premenstrual syndromes or the like, which make use of γ-linolenic acid show their desired effects if patients continuously ingest them in advance over a long period of time and therefore, they are insufficient in the immediate effect. More specifically, they suffer from such a problem that if the patients ingest them after the appearance of such symptoms, they cannot effectively mitigate the symptoms and they cannot meet the requirements of the patients. In the present invention, however, it has been found that the glycerin fatty acid ester, which carries γ-linolenic acid residues at 1- and/or 3-positions thereof, shows an effect of mitigating the symptoms associated with premenstrual syndromes and that the ester shows an excellent immediate effect. In this respect, the glycerin fatty acid ester may be a monoglycerin fatty acid ester, a diglycerin fatty acid ester or a triglycerin fatty acid ester insofar as it has γ-linolenic acid residues at 1- and/or 3-positions thereof. Diglycerin fatty acid esters and monoglycerin fatty acid esters are rather preferably used in the present invention, while taking note of the absorbability of the resulting drug. In addition, the glycerin fatty acid esters in which γ-linolenic acid residues are linked at the 2-position are also preferred in the present invention. This is a preferred embodiment since such an embodiment permits the improvement of the overall effect of mitigating the symptoms associated with premenstrual syndromes.

More preferably, the glycerin fatty acid ester of the present invention carries at least one fatty acid residue having 2 to 12 carbon atoms since such an embodiment would further improve the immediate effect thereof in the mitigation of the symptoms associated with PMS. In this connection, the term “fatty acid residue” used herein means a group obtained by removing the OH group from the carboxyl group of the corresponding fatty acid.

Fatty acid residues having 2 to 12 carbon atoms comprise those derived from short chain fatty acids having 2 to 5 carbon atoms and middle chain fatty acids having 6 to 12 carbon atoms, in particular, those derived from saturated fatty acids. Examples of middle chain fatty acids are caproic acid, caprylic acid, capric acid and lauric acid. In the present invention, fatty acid residues may preferably be those derived from fatty acids having 6 to 12 carbon atoms, in particular, saturated fatty acids having 6 to 12 carbon atoms. More preferably, the fatty acid residues are those derived from fatty acids having 8 to 12 carbon atoms, in particular, saturated fatty acids having 8 to 12 carbon atoms. Particularly preferred fatty acid residues are those derived from caprylic acid and/or capric acid.

The foregoing glycerin fatty acid esters can be prepared by, for instance, transesterifying fats and oils in which γ-linolenic acid is incorporated or subjecting fats and oils containing triglycerin fatty acid esters, to which γ-linolenic acid is linked at the 2-position thereof, to a chemical or enzymatic transesterification treatment, as will be detailed later. Moreover, the foregoing reaction solution can be concentrated and/or purified by, for instance, column chromatography technique to give a solution having a high concentration.

The term “immediate efficacy” used herein means that a particular drug immediately shows a desired effect of mitigating the PMS symptoms, which appear in and/or during the corpus luteum-activated phase, when a patient does not ingest the drug prior to the corpus luteum-activated phase, but ingests in and/or during the corpus luteum-activated phase. In this connection, the term “corpus luteum-activated phase” in general means the term of about two weeks corresponding to the high body temperature phase prior to the menstruation.

For instance, the glycerin fatty acid ester according to the present invention can suitably be incorporated into, for instance, the following drug for mitigating the symptoms associated with premenstrual syndromes, a food or drink for mitigating the symptoms associated with premenstrual syndromes and a fats and oils-containing composition for mitigating the symptoms associated with premenstrual syndromes. The glycerin fatty acid ester is excellent in the solubility in oil and therefore, it is preferred to use the same by incorporating it into, for instance, fats and oils while taking into consideration the usability thereof or the like.

In addition, the present invention also relates to a drug for mitigating the symptoms associated with premenstrual syndromes, which comprises, as an effective component, the foregoing glycerin fatty acid ester for mitigating the symptoms associated with premenstrual syndromes. Preferably, the drug for mitigating the symptoms associated with premenstrual syndromes further comprises at least one member selected from the group consisting of vitamin B's, vitamin E, herbs and mineral salts. Moreover, it is also possible to use the fats and oils-containing compound for mitigating the symptoms associated with PMS according to the present invention in combination with known functional materials, which are effective for mitigating the symptoms associated with PMS. Examples of such functional materials are mineral salts such as calcium, magnesium, zinc and iron salts; vitamins such as vitamin B₁, B₂, B₆, B₁₂, folic acid and vitamin E; herbs such as European Hypericum erectum, safflower, saffron and lemon burm as well as extracts thereof. These functional materials may be used in the form of tablets or soft capsules by adding them to the foregoing general processed foods. Furthermore, if a proper quantity of a drug for mitigating the PMS symptoms according to the present invention is continuously administered to a patient, it would be expected to enjoy an effect of reducing the lipid concentration in the blood.

In this connection, the drug for mitigating the PMS symptoms may comprise, for instance, at least one member selected from the group consisting of ginkgo leaves, passionflower, lemon burm and ginger, as the herb component. Moreover, the symptom associated with premenstrual syndromes comprises a plurality of symptoms and therefore, the drug may comprise herbs suitably used for the mitigation of these symptoms. For instance, when it is intended to mitigate the symptoms caused by hemokinesis-inhibition, the drug preferably comprises, as the herb component, at least one member selected from the group consisting of ginkgo leaves, passionflower and ginger. When it is intended to mitigate the positive psychic symptoms, the drug preferably comprises, as the herb component, at least one member selected from the group consisting of ginkgo leaves, passionflower and ginger. When it is intended to mitigate the symptoms of digestive system, the drug preferably comprises, as the herb component, at least one member selected from the group consisting of chamomile and lemon burm. When it is intended to mitigate the symptoms associated with negative psychic syndromes, the drug preferably comprises, as the herb component, at least one member selected from the group consisting of chamomile and lemon burm. When it is intended to mitigate the dolorific symptoms, the drug preferably comprises, as the herb component, at least one member selected from the group consisting of feverfew and rosemary. When it is intended to mitigate the easy susceptibility to fatigue, the drug preferably comprises, as the herb component, at least one member selected from the group consisting of feverfew and rosemary. When it is intended to mitigate the dropsy, the drug preferably comprises, as the herb component, at least one member selected from the group consisting of common dandelion and celery. When it is intended to mitigate the reduction of the ability to concentration, the drug preferably comprises, as the herb component, at least one member selected from the group consisting of common dandelion and celery. Further, when it is intended to mitigate the symptoms of mastalgia, the drug preferably comprises, as the herb component, at least one member selected from the group consisting of common dandelion and celery.

The agent for mitigating the symptoms associated with premenstrual syndromes according to the present invention may orally or parenterally be administered safely to human beings and animals in the form of, for instance, a drug or a quasi-drug. Examples of parenteral administration routes include intravenous injection, intra-arterial injection, intramuscular injection, subcutaneous injection, endermic injection, intraperitoneal injection, intraspinal injection, peridural injection, percutaneous (transdermal) administration, administration through lung, pernasal administration, perintestinal administration, administration through the oral cavity and permucosal administration. In addition, examples of the dosage forms thereof are injections, suppositories (such as rectal, urethral and vaginal suppositories), liquids for external use (such as injections, gargles, mouth washes, fomentations, inhalants, sprays, aerosols, enema, paints, cleaning-wiping agents, disinfectants, nasal drops and ear drops), cataplasms, percutaneous absorption tapes, external preparations for the skin; and ointments (such as pastes, liniments and lotions). In addition, examples of pharmaceutical preparations for oral administration include tablets for internal use (such as uncoated (naked) tablets, sugar-coated tablets, coating tablets, enteric coated tablets and chewable tablets); tablets administered through the oral cavity (such as buccal tablets, sublingual tablets, troche tablets and adhesive tablets); powders; capsules (such as gelatin capsules, hard capsules and soft capsules); and granules (such as coated granules, pills, troches, liquid preparations, or pharmaceutically acceptable sustained release preparations thereof). Examples of liquid preparations for oral administration include mixtures for internal use, shake mixtures, suspensions, emulsions, syrups, dry syrups, elixirs, infusions, decoctions and lemonades.

These pharmaceutical preparations can be prepared according to any known manufacturing techniques and therefore, can be administered to patients in the form of pharmaceutical compositions, which comprise pharmaceutically acceptable additives such as bases, carriers, vehicles (or excipients), disintegrators, lubricants and coloring agents.

Examples of carriers and vehicles used in these pharmaceutical preparations are lactose, glucose, sucrose, mannitol, potato starch, corn starch, calcium carbonate, calcium phosphate, calcium sulfate, crystalline cellulose, powdery licorice and gentian powder.

Examples of binders used in these pharmaceutical preparations are starches, tragacanth gum, gelatin, syrup, polyvinyl alcohol, polyvinyl ether, polyvinyl pyrrolidine, hydroxypropyl cellulose, methyl cellulose, ethyl cellulose and carboxymethyl cellulose. Examples of disintegrators used in these pharmaceutical preparations are starches, agar, gelatin powder, sodium carboxymethyl cellulose, calcium carboxymethyl cellulose, crystalline cellulose, calcium carbonate, sodium hydrogen carbonate and sodium alginate. Examples of lubricants used in these pharmaceutical preparations are magnesium stearate, talc, hydrogenated plant oils and macrogol. The coloring agents used in these pharmaceutical preparations may be pharmaceutically acceptable ones. In addition, if preparing an injection, additives such as a pH adjustor, a buffering agent, a stabilizer and/or a plasticizer may, if necessary, be added in addition to the foregoing ingredients to thus prepare each injection according to the usual method.

In case where tablets and granules are prepared, they may be coated with sugar, gelatin, hydroxypropyl cellulose, purified shellac, gelatin, glycerin, sorbitol, ethyl cellulose, hydroxypropyl cellulose, hydroxypropyl methyl cellulose, polyvinyl pyrrolidine, cellulose phthalate acetate, hydroxypropyl methyl cellulose phthalate, methyl methacrylate and methacrylic acid polymers, or they may be covered with at least two layers. Moreover, they may be encapsulated in capsules of, for instance, ethyl cellulose or gelatin.

The dosage forms of pharmaceutical preparations for external use may be solid, semi-solid, semi-solid-like or liquid pharmaceutical preparations for percutaneous, permucosal administration such as administration through the oral cavity or pernasal administration.

Liquid pharmaceutical preparations may be, for instance, emulsions or emulsoids such as pharmaceutically acceptable emulsions or lotions; tinctures for external use; and liquid preparations for permucosal administration. These pharmaceutical preparations may comprise, for instance, commonly used diluents such as ethanol, oil components and emulsifying agents.

Examples of semi-solid pharmaceutical preparations are ointments such as oily ointments and hydrophilic ointments. These semi-solid pharmaceutical preparations may comprise, for instance, water, vaseline, polyethylene glycol, oil components and surfactants, as commonly used bases or carriers.

Examples of semi-solid and solid pharmaceutical preparations are pastes for percutaneous administration or permucosal administration (such as administration through the oral cavity and pernasal administration), for instance, (hard) plasters (such as rubber plasters and plasters), films, tapes or cataplasms. These pharmaceutical preparations may comprise, as commonly used bases and/or carriers, a rubbery polymer such as natural rubber and synthetic rubber such as butadiene rubber, SBR and SIS; gelatin; a slurry-forming agent such as kaolin and zinc oxide; a hydrophilic polymer such as sodium carboxymethyl cellulose and sodium polyacrylate; a tackifier such as acrylic resin and liquid paraffin; water; other oil components; and surfactants.

These pharmaceutical preparations may further comprise an auxiliary agent such as a stabilizer, a solubilizing agent and a promoter for percutaneous absorption; or other additives such as an aromatic and/or an antiseptic.

The passage “comprising the glycerin fatty acid ester of the present invention as an effective component” herein used means that the pharmaceutical preparation comprises the same in an amount required for the achievement of the desired effect and the content thereof in the preparation is not restricted to any specific range. It has been found that the daily dose of the γ-linolenic acid moieties linked at the 1-, 3-positions is not less than 10 mg, preferably not less than 30 mg and more preferably not less than 50 mg, on the basis of the results obtained through several times of ingestion tests in which the drug containing the foregoing ester is administered to patients, as subjects, suffering from the PMS symptoms, while variously changing the amount of γ-linolenic acid. It has also been found that the total amount of γ-linolenic acid is desirably not less than 100 mg, as a standard. However, the dose may vary depending on various conditions such as age, sex, body weight, the seriousness of the symptoms and the health condition of the patient and the total amount of γ-linolenic acid is not necessarily limited to the foregoing range.

Moreover, the content of the glycerin fatty acid ester according to the present invention present in the drug for mitigating the symptoms associated with PMS preferably falls within the range, which can ensure the foregoing intake. Although the content cannot unconditionally be determined since it may vary depending on, for instance, the mass, size, mode of administration, unit dose and frequency of the administration of each particular pharmaceutical preparation, it is, for instance, not less than 0.01% by mass, preferably not less than 0.1% by mass, more preferably not less than 1% by mass, particularly preferably not less than 10% by mass, most preferably not less than 40% by mass and particularly most preferably 50 to 100% by mass.

The present invention further relates to a food or drink for mitigating the symptoms associated with the PMS comprising the foregoing glycerin fatty acid ester for mitigating the symptoms associated with the PMS, which has an excellent immediate effect. To obtain such a food or drink for mitigating the symptoms associated with the PMS, it is possible to directly incorporate the glycerin fatty acid ester of the present invention or the foregoing drug for mitigating the symptoms associated with the PMS into a desired food or drink.

In addition, the food or drink of the present invention may further comprise other physiologically active components to improve various functions of the consumers. Examples of such components are antioxidants, oily components, and a variety of vitamins, minerals and/or amino acids for the nutrition-enrichment.

The antioxidant components are not restricted to any particular one, but specific examples thereof include tocopherols and derivatives thereof; tocotrienols and derivatives thereof; lignans such as sesamin, episesamin, sesaminol, sesamolin and sesamol as well as glycosides thereof; carotenoids such as β-carotene and derivatives thereof; tannins such as gallic acid and ellagic acid as well as derivatives thereof; flavonoids such as flavone, catechin, quelcetin and leucoanthocyanidin; quinones such as ubiquinone and vitamin K; ferulic acid derivatives such as oryzanol; and olive extract.

Examples of oily components are animal fats and oils such as lard, beef tallow and cream in milk; fats and oils derived from marine products such as whale oil and herring oil; and vegetable fats and oils such as soybean oil, rape seed oil, cotton seed oil, rice oil, corn oil, sesame oil, peanut oil, sunflower oil, safflower oil, camellia oil, olive oil, linseed oil, tung oil, castor oil, coconut oil, palm oil and cacao butter, but they are not restricted to any particular one at all. Examples thereof further include naturally occurring ones or those obtained through chemical reactions or enzymatic reactions, such as MCT, MLCT, diglycerides and monoglycerides; and structure-modified fats and oils in which the structures of fatty acid moieties are specially designed.

The components for the nutrition-enrichment such as vitamins, minerals and amino acids are not particularly limited to specific ones, but it is desirable to use those defined and specified in “Official Formulary of Food Additives”.

Moreover, it is also possible to use or incorporate, into the food and drink according to the present invention, ingredients (or raw materials) commonly used in the usual foods and drinks. Such ingredients are not restricted to any particular one, but specific examples thereof are a variety of seasonings such as miso, soy sauce, sauce, ketchup, bouillon, soup for roast meat, roux for curry, and premixes for stew and soup as well as soup stock; or the like.

The foods and drinks are not particularly limited in, for instance, shapes, but specific examples thereof include confectionery, drinks, processed foods, retort foods, various kinds of seasonings, cooked rice or the like, fats and oils, fats and oils-containing compositions, processed fats and oils-containing products, a variety of foods for cooking in a microwave oven, frozen foods and health foods and drinks as well as enriched foods and drinks.

Specific examples of the foods and drinks according to the present invention will be listed below, but the present invention is not restricted to these specific examples at all. The foods and drinks according to the present invention are not limited in, for instance, shapes and specific examples thereof are Japanese-style confections such as Okaki, Japanese crackers (rice crackers), millet-and-rice cakes, buns with a bean-jam filling and starch jelly; a variety of European confectionery such as cookies, biscuits, crackers, pies, castilla, doughnuts, custard pudding, sponge cakes, waffle, butter cream, custard cream, cream puff, chocolate, confectionery with chocolate, caramel, candy, chewing gum, jelly, hot cake, bread and bun; snack confectionery such as potato chips; frozen confectionery such as ice cream, ice candy and sherbet; refreshing beverages such as lactic acid beverage, lactobacillus-fermented beverage, concentrated dairy beverage, fruit juice beverage, fruit flesh-containing beverage, functional beverage and carbonated beverage; table luxuries such as green tea, black tea, coffee and cocoa; dairy products such as beverages containing the same, fermented milk, processed milk and cheese; processed soybean foods such as soy milk and soybean card; jam; fruits dipped in syrup; pastes such as flower paste, peanut paste and fruit paste; pickles and salted vegetables; products derived from grains such as wheat vermicelli and pasta; (beast) meat products such as ham, sausage, bacon, dry sausage, beef jerky and hamburg; sea foods such as fish meat ham, fish meat sausage, boiled fish paste, Chikuwa (a kind of fish paste) and cakes of pounded fish; dried fishes and shellfishes; dried bonito, horse mackerel and mackerel; salted guts of sea urchins and cuttlefishes; dried Mirin-seasoned dried cuttlefishes and fishes; foods boiled down in soy sauce using, for instance, laver, small fishes, shellfishes, shiitake (Cortinellus shiitake), edible wild plants and Kombu (sea tangle); retort foods such as curry and stew; a variety of seasonings such as miso, soy sauce, sauce, ketchup, bouillon, soup for roast meat, roux for curry, and premixes for stew and soup as well as soup stock; cooked rice or the like (grains); fats and oils and fats and oils-containing compositions comprising fats and oils and lecithin, plant's sterols, tocopherols, β-carotene and/or vitamin C; processed fats and oils-containing products such as margarine, shortenings, mayonnaise and dressings; and a variety of frozen foods and those used for cooking in a microwave oven, which comprise fats and oils. In particular, preferred are, for instance, cooked rice, a variety of seasonings and fats and oils as well as processed fats and oils-containing products such as margarine, shortenings, mayonnaise and dressings, since the consumers would ingest these foods and drinks continuously. Moreover, these foods and drinks are not restricted in, for instance, their shapes and may be in the form of, for instance, a solid-like, semi-solid-like, gel-like, liquid-like and powdery shapes. When they are used as health foods or drinks or enriched foods or drinks, they may be in the form of, for instance, tablets, capsules, liquid preparations and granules.

In the present invention, the foods and drinks for mitigating the symptoms associated with the PMS are preferably enriched foods and drinks for health. Moreover, the glycerin fatty acid ester according to the present invention is excellent in the solubility in oil and therefore, the foods and drinks of the present invention are preferably in the form of fats and oils-containing compositions and/or processed fats and oils-containing products. In this respect, the term —fats and oils-containing composition” herein used means a composition comprising fats and oils or a composition containing fats and oils and a variety of effective components for the improvement of a variety of functions or such a composition further subjected to a chemical treatment. In other words, the composition is similar to that commonly referred to as “fats and oils”. Moreover, the term “processed fats and oils-containing product” herein used means a food or drink obtained by processing fats and oils, such as margarine, dressings and mayonnaise listed above.

In addition, in the edible fats and oils-containing composition, which comprises the foregoing glycerin fatty acid ester for mitigating the symptoms associated with the PMS, the amount of the γ-linolenic acid residues linked at the 1-, 3-positions or the total amount of the γ-linolenic acid residues present in the substituents R₁ and R₃ in the foregoing chemical formula (I) is preferably not less than 1% by mass, preferably not less than 2% by mass, more preferably not less than 5% by mass, particularly preferably not less than 10% by mass, most preferably not less than 30% by mass and particularly most preferably 40 to 90% by mass on the basis of the total amount of the constituent fatty acid residues. Inasmuch as the foregoing requirement for the content of the γ-linolenic acid residue is satisfied, the foregoing composition can preferably be used as a raw material for foods and drinks or used in the cooking of foods and drinks or further used as the foregoing drug for mitigating the symptoms associated with the PMS syndromes and the composition can thus effectively show its effect, in particular, the immediate effect of mitigating the symptoms associated with the PMS.

Moreover, the ratio of the amount of γ-linolenic acid residues linked at the 1-, 3-positions to that of the residues linked at the 2-position or the ratio of the total amount of the γ-linolenic acid residues present in the substituents R₁ and R₃ of the foregoing chemical formula (I) to that of the residues present in the substituent R₂ of the chemical formula ranges from 1:0.1 to 1:3, preferably 1:0.5 to 1:3, more preferably 1:0.5 to 1:2 and most preferably 1:0.5 to 1:1.5 on the basis of the total amount of the constituent fatty acid residues. In this case, the resulting composition shows the immediate effect of the foregoing fats and oils-containing composition for mitigating the symptoms associated with the PMS as well as the long-acting effect and therefore, the composition preferably shows, as a whole, excellent effect of mitigating the symptoms associated with the PMS.

In addition, in the foregoing fats and oils-containing composition for mitigating the symptoms associated with the PMS, the total amount of the γ-linolenic acid residues or that of the γ-linolenic acid residues present in the substituents R₁, R₂ and R₃ of the foregoing chemical formula (I) is preferably not less than 1% by mass, preferably not less than 2% by mass, preferably not less than 5% by mass, more preferably not less than 10% by mass, particularly preferably not less than 30% by mass and particularly most preferably 40 to 90% by mass on the basis of the total amount of the constituent fatty acid residues. If the daily dose is considered to be 10 g in case where the fats and oils are directly used for cooking, they should comprise the γ-linolenic acid residues in a content of not less than 1% by mass in order to ensure the effect of mitigating the symptoms associated with the PMS, while if they are used as a pharmaceutical preparation by directly encapsulating them in a capsule, the content thereof is preferably not less than 5% by mass in the light of the usual dose thereof.

Further, the total amount of the fatty acid residues having 2 to 12 carbon atoms, or that of the fatty acid residues having 2 to 12 carbon atoms present in the substituents R₁, R₂ and R₃ of the foregoing chemical formula (I) is preferably not less than 5% by mass, preferably 5 to 30% by mass and more preferably 10 to 25% by mass on the basis of the total amount of the constituent fatty acid residues. In this case, the absorbability of the γ-linolenic acid is improved and this in turn improve the immediate effect of mitigating the symptoms associated with the PMS.

Furthermore, the rate of the long chain saturated fatty acids or the total amount of the long chain saturated fatty acids present in the substituents R₁, R₂ and R₃ of the foregoing chemical formula (I), based on the total amount of the long chain fatty acids constituting the fats and oils-containing composition, is preferably not more than 20% by mass, more preferably not more than 15% by mass and further preferably not more than 7% by mass. If the rate exceeds 20% by mass, the stability of the composition is lowered, crystals of the fats and oils are precipitated in the composition and therefore, the composition is in general unfavorable for eating it as it is or uncooked state.

In the present invention, it is preferred that the glycerin fatty acid ester comprises fatty acid moieties having 2 to 12 carbon atoms in a content as high as possible as the constituent fatty acid moieties. In this respect, the highest absorbability can be anticipated when three fatty acid moieties having 2 to 12 carbon atoms are linked to glycerin per molecule, but good absorbability can be ensured when one or two such fatty acid moieties are linked to glycerin per molecule.

As has been discussed above in detail, the fatty acids having 2 to 12 carbon atoms are preferably those having 6 to 12 carbon atoms, in particular, saturated fatty acids having 6 to 12 carbon atoms and more preferably fatty acids having 8 to 10 carbon atoms, in particular, saturated fatty acids having 8 to 10 carbon atoms, with caprylic acid and/or capric acid being particularly preferred. In case where the composition is used as a food or drink or orally administered, the short chain fatty acids are unfavorable from the viewpoint of the flavor and therefore, there is such a tendency that fatty acids having a relatively large number of carbon atoms are favorably used as the fatty acid moieties. In this connection, the term “long chain fatty acid residues” other than the foregoing short chain and middle chain fatty acid residues herein means those derived from fatty acids having not less than 14 carbon atoms, preferably those derived from saturated and unsaturated fatty acids having 14 to 24 carbon atoms. Specific examples of long chain fatty acids having not less than 14 carbon atoms are long chain saturated fatty acids such as myristic acid, palmitic acid, stearic acid, arachidic acid, behenic acid, lignoceric acid and cerotic acid; and long chain unsaturated fatty acids such as myristoleic acid, pentadecenoic acid, palmitoleic acid, hexadecatrienoic acid, heptadecenoic acid, oleic acid, linolenic acid, linolenic acid, γ-linolenic acid, octadecatetraenoic acid, icosenoic acid, icosadienoic acid, icosatrienoic acid, icosatetraenoic acid, arachidonic acid, icosapentaenoic acid, docosenoic acid, docosadienoic acid, docosapentaenoic acid and docosahexaenoic acid.

As the foregoing edible fats and oils-containing composition for mitigating the symptoms associated with the PMS, preferred are those obtained through transesterification. More specifically, preferred are those obtained by subjecting, to transesterification, fats and oils comprising 5 to 95% by mass, preferably 10 to 95% by mass, more preferably 15 to 95% by mass and particularly preferably 50 to 95% by mass of γ-linolenic acid. In particular, preferred are those obtained through transesterification of fats and oils comprising γ-linolenic acid in an amount falling within the range defined above and 5 to 40% by mass, preferably 5 to 30% by mass and more preferably 10 to 20% by mass of fatty acids having 2 to 12 carbon atoms. In this respect, the foregoing content is calculated while taking account of the fatty acids per se and those present in the glycerin fatty acid ester in the form of fatty acid residues. Examples of raw materials are not restricted to particular ones and include various kinds of animal fats and oils, vegetable fats and oils, purified fats and oils such as diglycerides, monoglycerides and MCT, processed fats and oils and γ-linolenic acid and fatty acids such as those having 2 to 12 carbon atoms, which are used alone.

In particular, there may be listed such currently used edible oils having a relatively high content of γ-linolenic acid as fats and oils derived from plants [for instance, oils derived from evening primrose (a plant belonging to the genus Oenothera)=evening primrose oil] and Borage officinalis (a plant belonging to the genus Boraginaceae); microorganism-fermented fats and oils such as those derived from filamentous fungi, microorganisms belonging to Cunninghamella and Choanephora); these fats and oils whose degree of unsaturation is reduced by the quality improvement, and hydrogenated and fractionated products thereof.

In addition, the following substances (A) and/or (B) can be prepared for use as a starting material and then subjected the starting material to transesterification:

(A): At least one member selected from the group consisting of borage oil, evening primrose oil and microorganism-fermented fats and oils; and

(B): At least one member selected from the group consisting of fatty acids each having 2 to 12 carbon atoms, glycerin fatty acid esters carrying 1 to 3 fatty acid residues having 2 to 12 carbon atoms and fats and oils containing the same.

As the methods and conditions for the transesterification, there can be used either a chemical method using a catalyst or a biological method, which makes use of an enzyme. Examples of enzymes for decomposing lipids (lipases) include lipases derived from microorganisms belonging to the genus Alcaligenes, Candida, Rhizopus, Mucor or Pseudomonas; and phospholipase A derived from liver, with the lipases derived from microorganisms belonging to the genus Candida or Rhizopus being particularly preferred.

For instance, if sodium methylate is used as such a catalyst, it is preferred to carry out the transesterification at ordinary pressure in a nitrogen gas stream or at a reduced pressure on the order of not more than 10 Pa and at a temperature ranging from 80 to 120° C., for 10 to 60 minutes.

More specifically, the transesterification, which makes use of sodium methylate as a catalyst, can be performed by heating a mixture of raw materials to 80 to 120° C. at a reduced pressure of not more than 100 Pa to thus remove the gaseous components and moisture present in the raw mixture, adding 0.02 to 0.5% by mass of sodium methylate to the raw mixture and then stirring the resulting mixture at ordinary pressure in a nitrogen gas stream or at a reduced pressure on the order of not more than 10 Pa and at a temperature ranging from 80 to 120° C., for 10 to 60 minutes. The end point of the reaction can be confirmed by monitoring the composition of triglycerides as reaction products according to the gas chromatography technique. The reaction can be quenched by addition of water or an acid such as phosphoric acid. Thereafter, the reaction product is sufficiently washed to remove the catalyst and the excess of acid, followed by drying and discoloration and deodorization of the product.

Moreover, when a lipid is used, the transesterification is preferably carried out at a temperature ranging from 40 to 100° C. for 2 to 48 hours. The kind of the enzyme to be used may appropriately be selected depending on the reaction conditions. For instance, if a raw material comprising fats and oils, in which only γ-linolenic acid is present, is prepared by transesterification, an enzyme specific to the 1-, 3-positions can be used and if the raw material selected comprises those carrying γ-linolenic acid residues at the 2-position, which are abundantly present in the naturally occurring fats and oils, preferably used herein are enzymes, which can randomly transesterify the raw material.

More specifically, if the transesterification is performed using a lipase, the temperature of the raw material (or reaction temperature) should be adjusted to the range of from 40 to 100° C. in which the lipase shows its activity satisfactorily. Then such a lipase is added to the raw material in a rate ranging from 0.005 to 10% by mass on the basis of the total weight of the raw material and the transesterification is carried out over 2 to 48 hours. This reaction is desirably carried out at ordinary pressure in a nitrogen gas stream. The end point of the reaction can be confirmed by monitoring the composition of triglycerides as reaction products according to the gas chromatography technique. The reaction can be quenched by removing the enzyme through filtration. The reaction product is washed with water, followed by drying and then discoloration and deodorization of the same according to the usual methods. In this connection, if middle chain fatty acids are used as raw materials, free fatty acids are removed using a thin film type evaporator after quenching the reaction.

If the transesterification using a lipase is insufficient, the rate of the triglycerides carrying three middle chain fatty acid residues in the molecule increases. A fats and oils-containing composition having a high content of such triglycerides carrying three middle chain fatty acid residues in the molecule is characterized in that it has a low body fat-accumulating ability, but it is insufficient in the cooking and processing properties.

Although we have already discussed above as to the middle chain fatty acids included in the fatty acids having 2 to 12 carbon atoms, middle chain fatty acid triglycerides may be used instead of or in combination with the middle chain fatty acids. Such middle chain fatty acid triglycerides may be, for instance, triglycerides obtained by esterifying the foregoing middle chain fatty acids with glycerin according to the usual method, but examples thereof are in general single fatty acid residue-containing or mixed fatty acid residue-containing triglycerides usually called MCT (medium chain triglycerides) constituted by saturated fatty acids having 8 to 10 carbon atoms such as fatty acids derived from coconut oil. For instance, preferably used include triglycerides of caprylic acid/capric acid (=60˜75/25˜40 (mass ratio)).

The rates of γ-linolenic acid, middle chain fatty acids and γ-linolenic acid linked to the 1-, 3-positions of the triglyceride with respect to the overall fatty acids constituting the triglyceride; the rate of the triglycerides carrying three middle chain fatty acid residues with respect to the total triglycerides constituting the fats and oils-containing composition; and if necessary, the rate of the long chain saturated fatty acids with respect to the total long chain fatty acids constituting the fats and oils-containing composition can be controlled by adjusting the mixing ratio of the raw fats and oils to the middle chain fatty acids and determining or monitoring the triglyceride composition of the reaction product obtained during the transesterification reaction, while taking into consideration the composition of the raw fats and oils.

The fats and oils-containing composition of the present invention can likewise be extracted from plants, which have been subjected to plant breeding by a gene recombination technique so that the plant produces the fats and oils-containing composition according to the present invention and examples of such plants are evening primrose, Borage officinalis, soybean, ripe seed, corn, coconut palm, palm, olive, linseed, sesame, rice, sunflower, safflower, camellia, cotton seed and KUHEA.

Edible fats and oils can be added to the fats and oils-containing composition of the present invention prepared by the foregoing methods. Examples of such edible fats and oils are vegetable fats and oils derived from, for instance, evening primrose, Borage officinalis, soybean, ripe seed, corn, coconut palm, palm, olive, linseed, sesame, rice, sunflower, safflower, camellia, cotton seed and KUHEA and hydrogenated fats and oils prepared from these vegetable fats and oils; animal fats and oils such as fish oils containing EPA and DHA, lard and beef tallow and hydrogenated fats and oils prepared from these animal fats and oils.

The edible fats and oils-containing composition according to the present invention can be used as a fats and oils-containing composition for cooking, as it is or after blending with additives commonly used in such a composition for cooking. Moreover, the fats and oils-containing composition according to the present invention, prepared according to the foregoing methods, can likewise be used as a fats and oils-containing composition for cooking, as it is or after blending with additives commonly used in such a composition for cooking. Examples of such additives are polyglycerin fatty acid esters, sucrose fatty acid esters, sorbitan fatty acid esters, vitamin E, ascorbic acid fatty acid esters, lignan, coenzyme Q, phospholipids, oryzanol and diglycerides for the improvement of storage stability, stability to oxidation, heat stability and for the inhibition of crystallization upon cooking; and vitamin E, ascorbic acid fatty acid esters, lignan, coenzyme Q, phospholipids and oryzanol for expecting the achievement of a geriatric disease preventive effect, an effect of preventing diseases originated from the habit of life, endogenous oxidation-inhibitory effect and the obesity-inhibitory effect.

In this respect, if the foregoing edible fats and oils-containing composition for mitigating the symptoms associated with the PMS is used in the applications in which the composition is not cooked by heating, for instance, it is used as a raw material for preparing pharmaceutical preparations packed in gelatin capsules or for preparing ice cream, there may be used an edible fats and oils-containing composition in which the rate of the triglycerides carrying three fatty acid residues having 2 to 12 carbon atoms in the molecule with respect to the overall triglycerides constituting the edible composition is not less than 10% by mass without any problem. On the other hand, if the fats and oils-containing composition of the present invention is used in applications wherein it is cooked by heating, the rate is preferably not more than 10% by mass, more preferably not more than 3% by mass and particularly preferably not more than 1% by mass. If the rate exceeds 10% by mass, the degrees of fuming and/or foaming observed during the cooking by heating increase and accordingly, the application of the composition as fats and oils is quite limited. In this respect, if the rate is less than 1% by mass, there are observed considerable improvement in the effects of inhibiting fuming and/or foaming.

The fats and oils-containing composition according to the present invention, which has an effect of mitigating the PMS symptoms and a low ability of body fat-accumulation can be prepared by appropriately blending fats and oils having a high content of γ-linolenic acid residues with, for instance, fats and oils containing fatty acids having 2 to 12 carbon atoms; and then subjecting the resulting blend to transesterification in the presence of sodium methylate as a catalyst or a lipase in such a manner that the rate of the fatty acids having 2 to 12 carbon atoms with respect to the total fatty acids constituting the fats and oils-containing composition falls within the range defined above.

In the foregoing transesterification reaction, if the rate of the triglycerides having three middle chain fatty acid residues with respect to the total triglycerides constituting the composition and/or the rate of the long chain saturated fatty acid with respect to the total long chain fatty acids constituting the composition are controlled so as to fall within the foregoing ranges defined above respectively, in addition to the foregoing adjustment, a preferred fats and oils-containing composition can be prepared, which possesses the desired PMS-mitigating effect, has a low ability of body fat-accumulation and is excellent in stability.

The foregoing edible fats and oils-containing composition for mitigating the symptoms associated with the PMS according to the present invention can likewise be preferably incorporated into the drug for mitigating the symptoms associated with the PMS as well as the food or drink for mitigating the symptoms associated with the PMS.

It is a matter of course that the edible fats and oils-containing composition for mitigating the symptoms associated with the PMS prepared according to the foregoing methods can be packed in or processed in the form of a soft or hard capsule widely used in the nutrition-enriched foods prior to the practical ingestion. At this stage, it is possible to use, if necessary, an emulsifying agent such as a sucrose fatty acid ester or beeswax and a viscosity-controlling agent. Moreover, the edible composition is excellent in quality stability and taste and texture and therefore, it can directly be ingested, when it is processed into powdery fats and oils or fats and oils in a liquid emulsion, while it contains a large amount of unsaturated fatty acids, or can indirectly be ingested, when these powdered or emulsified fats and oils are further processed or incorporated into general foods. Examples of such general foods to which the edible composition is applied are confectionery such as cookies, biscuits, cakes, chocolate and GUMI; beverages such as fruit juice-containing beverages, nutrition-enriched drinks and sports drinks; or seasoned or processed foods such as dressings, mayonnaise and margarine.

Moreover, the fats and oils-containing composition according to the present invention for mitigating PMS symptoms possesses taste and texture identical or superior to those observed for the usual and commercially available edible oils such as rape seed oil, corn oil, safflower oil and soybean oil and thus it can directly be used in the cooking of frizzled foods, foods fried in oil and mariner. Moreover, the degree of oil sputtering observed during the preparation of fries is identical to or lower than the usual edible oil.

It would be quite important that the effect of mitigating the symptoms associated with the PMS is appropriately evaluated to thus provide a drug for mitigating the symptoms associated with the PMS, a food or drink for mitigating the symptoms associated with the PMS, and an edible fats and oils-containing composition for mitigating the symptoms associated with the PMS, which can show such a desired mitigation effect.

For instance, the PMS symptoms are divided into not less than 2 groups so that each group consists of symptoms, which are liable to be simultaneously caused, and herbs or the like, which are effective for mitigating the PMS symptoms, can be used in combination for each group. In this connection, the term “division into not less than 2 groups” means that the symptoms are divided into not less than two groups such that each group consists of symptoms, which are apt to be caused in the same patient or in the patients belonging to the same generation.

As a result, the inventors of this invention have found that the PMS symptoms can be divided into at least two groups, preferably 2 to 10 groups, more preferably 2 to 6 groups and most preferably 3 to 5 groups depending on the types of patients (or factors such as physical constitutions, patients' living environments and patients' generations). For instance, the PMS symptoms can be divided into the following groups a to e wherein each group consists of symptoms liable to be simultaneously developed:

Group a: Symptoms associated with the hemokinesis-inhibition [such as stiffness in the shoulders and oversensitiveness to the cold]; positive psychic symptoms [such as touchiness, offensiveness or aggressiveness (including a psychological lift, a psychological uncontrollableness, a quarrel with others and use of strong languages against the family or friends), irritation, feeling tired of doing anything (including disagreeableness with the fact that she is a woman, feeling repugnance to the menstruation and unsociableness), sentimentalism, a rise in anxiety, loss of courage, love of solitude (including a desire of staying at home, such an impression that nobody understands or support her)] and mammary inflation;

Group b: Negative psychic symptoms [melancholy and a desire to rearrange something and put them in order], lumbagos, abdominal inflation, promotion of appetite, constipation, roughened skin and feeling of sleepiness;

Group c: Pains [abdominal pains and headaches], diarrhea, susceptibleness to the formation of acne, susceptibleness to fatigues, an increase in the discharge from the womb and enervation [including such a belief that she is worthless and inability to manage her health];

Group d: Dropsical swelling and the reduction of the ability to concentration [including decrease of efficiency and inability to get herself to work in her usual way]; and

Group e: Mastalgia.

In this respect, all of the symptoms classified into the same group may simultaneously be caused or one or at least two of them may simultaneously be caused. Moreover, one or at least two of other symptoms are added to the foregoing symptoms. In the foregoing, each symptom is expressed in terms of the typical one and therefore, symptoms expressed in terms of approximately synonymous expressions are likewise classified into the same group. For instance, approximately synonymous symptoms of the foregoing symptom “offensiveness or aggressiveness” include, for instance, a psychological lift, a psychological uncontrollableness, a quarrel with others and use of strong languages against the family or friends.

As to the foregoing Groups a to e, they can further be subdivided into subgroups according to, for instance, the following combinations to thus divide the PMS symptoms into at least two groups, preferably 2 to 10 groups, more preferably 2 to 6 groups and most preferably 3 to 5 groups.

For instance, the PMS symptoms can be divided into 5 groups a, b, c, d, and e; 4 groups such as groups a+b, c, d and e, groups a+c, b, d and e, groups a+d, b, c and e, groups a+e, b, c and d, groups a, b+c, d and e, groups a, b+d, c and e, groups a, b+e, c and d, groups a, b, c+d and e, groups a, b, c+e and d, and groups a, b, c and d+e; 3 groups such as groups a+b+c, d and e, groups a+b+d, c and e, groups a+b+e, c and d, groups a+c+d, b and e, groups a+c+e, b and d, groups a+d+e, b and c, groups a, b+c+d and e, groups a, b+c+e and d, groups a, b+d+e and c and groups a, b and c+d+e; and 2 groups such as groups a+d+e and b+c and groups a+d and b+c+e.

In addition, the symptoms belonging to each group can further be subdivided. For instance, the symptoms belonging to the group a can be subdivided into the following three groups:

Group a1: stiffness in the shoulders, touchiness, oversensitiveness to the cold, irritation and feeling tired of doing anything (including disagreeableness with the fact that she is a woman, feeling repugnance to the menstruation and unsociableness);

Group a2: mammary inflation, sentimentalism and a rise in anxiety;

Group a3: offensiveness or aggressiveness (including a psychological lift, a psychological uncontrollableness, a quarrel with others and use of strong languages against the family or friends), loss of courage and love of solitude (including a desire of staying at home and such an impression that nobody understands or support her).

The group b can be subdivided into the following two groups:

Group b1: lumbagos, promotion of appetite, roughened skin and feeling of sleepiness; and

Group b2: abdominal inflation, constipation, melancholy and a desire to rearrange something and put them in order.

The group c can be subdivided into the following two groups:

Group c1: abdominal pains, an increase in the discharge from the womb, susceptibleness to the formation of acne and headaches and

Group c2: diarrhea, susceptibleness to fatigues and enervation [including such a belief that she is worthless and inability to manage her health].

Thus, the PMS symptoms can be divided into 6 groups such as groups a1+a2, a3, b1, b2, c1 and c2; groups a1+a3, a2, b1, b2, c1 and c2; groups a2+a3, a1, b1, b2, c1 and c2; groups a1, a2, a3, b1+b2, c1 and c2 and groups a1, a2, a3, b1, b2 and c1+c2.

Moreover, the PMS symptoms can be divided into two groups such as groups a and b+c+d+e, groups b and a+c+d+e, groups c and a+b+d+e, groups d and a+b+c+e, groups e and a+b+c+d, groups a+b and c+d+e, groups a+c and b+d+e, groups a+d and b+c+e, groups a+e and b+c+d, groups b+c and a+d+e, groups b+d and a+c+e, groups b+e and a+c+d, groups c+d and a+b+e, groups c+e and a+b+d and groups d+e and a+b+c.

Among these groups, particularly preferred classification are, for instance, classification into 5 groups a, b, c, d and e; 4 groups a, b, c and d+e and 3 groups a+b+e, c and d.

In this respect, 29 typical PMS symptoms are shown in FIG. 1 as a dendritic diagram. In FIG. 1, the closer the positions of symptoms, the higher the probability of being simultaneously caused. For instance, the symptoms such as enervation and susceptibleness to fatigue are, in particular, apt to be caused simultaneously and subsequently, the symptoms such as diarrhea, headache and susceptibleness to the formation of acne are apt to be simultaneously caused and thus these symptoms are liable to be classified into the same group. In addition, the smaller the distance between two symptoms, the higher the probability of simultaneous outbreak of these symptoms. For instance, the probability of simultaneous outbreak of the symptoms: “touchiness” and “stiffness in the shoulders” is higher than that of the simultaneous outbreak of the symptoms: “an increase in the discharge from the womb” and “abdominal pains”.

If considering the aforementioned and other products such as pharmaceutical preparations, foods and beverages as the products for mitigating the PMS symptoms, examples of effective components are herbs such as the oil derived from evening primrose, the oil derived from borage, Aesculus Hippocastanum seeds, ginger (Zingiber officinale Roscoe), ginkgo leaves, Scent Jones wart, valerian, green tea, Centella asiatica Urban, EZOUKOGI (a plant belonging to the family Araliaceae), rosemary, scull cap, hops, passionflower, raspberry, common comfrey, garlic, SAILIUM, senna, olive, Japanese Mulberry, KASUKARASAGURADA, camomile, grape seeds, licorice, LUIBOSU, pine, lemon burm, Echinacea (coneflower), carrot, Chinese gutta percha, aloe, peppermint, kava, common comfrey, ginger, feverfew, black cohosh, common pomegranate, rosemary, catnip, aniseed, Chilean pepper, adlay, beefsteak plant's seeds, beefsteak plant's leaves, peppermint, Houttuynia cordata Thumb., Artemisia princeps Pump., red pepper, common dandelion, parsley, celery, artichoke, elder, Astragalus mongholicus Bunge, milk or holy thistle, common plantain, kava, gymnema, cat's-claw, cranberry, grape seeds, goldenseal, great burdock, May flower (English hawthorn), Chinese angelica, nettle, banaba, pumpkin seeds, bilberry, Chinese ephedra and Pausinystalia yohimbe; vitamins such as vitamin A, vitamin B₁, B₂, B₆, B₁₂, folic acid, vitamin C, vitamin D and vitamin E; and minerals such as calcium, iron, magnesium and zinc. These effective components or appropriate combination of at least two of them are selected so as to be adapted for each group to thus give each corresponding product.

For instance, the following combination or blend may be selected or used in order to obtain products adapted for the foregoing groups a, b, c and d+e:

Group a: The corresponding composition comprises at least one member selected from the group consisting of, for instance, oil derived from evening primrose, the oil derived from borage, Aesculus Hippocastanum seeds, ginger (Zingiber officinale Roscoe), ginkgo leaves, Scent Jones wart, valerian, green tea, Centella asiatica Urban, EZOUKOGI (a plant belonging to the family Araliaceae), rosemary, scull cap, hops, passionflower, raspberry, common comfrey, garlic, vitamin A, vitamin B₁, B₂, B₆, B₁₂, vitamin C, vitamin D and vitamin E;

Group b: The corresponding composition comprises at least one member selected from the group consisting of, for instance, the oil derived from evening primrose, the oil derived from borage, SAILIUM, senna, olive, Japanese Mulberry, KASUKARASAGURADA, camomile, grape seeds, licorice, LUIBOSU, garlic, pine, lemon burm, Echinacea (coneflower), carrot, Chinese gutta percha, aloe, peppermint, kava, common comfrey, ginger, vitamin A, vitamin B₁, B₂, B₆, B₁₂, folic acid, vitamin C, vitamin D and vitamin E;

Group c: The corresponding composition comprises at least one member selected from the group consisting of, for instance, the oil derived from evening primrose, the oil derived from borage, feverfew, black cohosh, common pomegranate, rosemary, catnip, aniseed, Chilean pepper, adlay, beefsteak plant's seeds, beefsteak plant's leaves, peppermint, Houttuynia cordata Thumb., Artemisia princeps Pump., raspberry, common comfrey, vitamin A, vitamin B₁, B₂, B₆, B₁₂, folic acid, vitamin C, vitamin D and vitamin E;

Group d+e: The corresponding composition comprises at least one member selected from the group consisting of, for instance, the oil derived from evening primrose, the oil derived from borage, carrot, red pepper, common dandelion, parsley, celery, vitamin A, vitamin B₁, B₂, B₆, B₁₂, folic acid, vitamin C, vitamin D and vitamin E.

Moreover, the symptoms associated with the PMS syndromes, whose outbreak frequency is high, can be classified into 2 to 8 groups depending on the generations of women to thus put, on the market, each product or composition for mitigating the symptoms, adapted for each classified generation or group. The number of groups thus classified is preferably 2 to 5 and more preferably 2 to 3. In this connection, the term “classification depending on the generations” herein used means that the symptoms can be classified into groups, each of which comprises symptoms observed for a particular generation. For instance, if the symptoms are divided into two groups depending on the generations of women, they can be divided into those peculiar to the women in the twenties and those peculiar to the women in the thirties, as will be detailed below. The basic symptoms commonly observed for the women in the both twenties and thirties include 0 or at least one member selected from the group consisting of abdominal pains, irritation, an increase in the discharge from the womb and feel of sleepiness and each group includes the following symptoms peculiar to each corresponding generation:

Symptoms Peculiar to Women in the Twenties: At least one symptom selected from the group consisting of mammary inflation, acne, lumbagos and promotion of appetite.

Symptoms Peculiar to Women in the Thirties: At least one symptom selected from the group consisting of headache and stiffness in the shoulders.

In addition, each symptom should include those expressed in terms of the synonyms thereof as has already been discussed above. In this respect, it is a matter of course that the women in the twenties include 20-year-old to 29-year-old women and also include 20˜29±5-year-old women because of the differences between individuals. It is likewise a matter of course that the women in the thirties include 30-year-old to 39-year-old women as well as 30˜39±5-year-old women for the same reason.

If considering foods and drinks, effective components adapted for each group are, for instance, as follows:

Components Adapted for Women in the Twenties: At least one member selected from the group consisting of, for instance, the oil derived from evening primrose, the oil derived from borage, passionflower, raspberry, common dandelion, olive, Japanese Mulberry, KASUKARASAGURADA, camomile, grape seeds, licorice, garlic, lemon burm, Echinacea (coneflower), carrot, Chinese gutta percha, aloe, peppermint, kava, common comfrey, ginger, beefsteak plant's seeds, beefsteak plant's leaves, Artemisia princeps Pump., adlay, Houttuynia cordata Thumb., rosemary, vitamin A, vitamin B₁, B₂, B₆, B₁₂, folic acid, vitamin C, vitamin D and vitamin E.

Components Adapted for Women in the Thirties: At least one member selected from the group consisting of, for instance, the oil derived from evening primrose, the oil derived from borage, Aesculus Hippocastanum seeds, ginger (Zingiber officinale Roscoe), ginkgo leaves, Scent Jones wart, valerian, green tea, EZOUKOGI (a plant belonging to the family Araliaceae), rosemary, scull cap, hops, passionflower, raspberry, garlic, camomile, lemon burm, feverfew, black cohosh, common pomegranate, rosemary, catnip, aniseed, Chilean pepper, celery, beefsteak plant's seeds, beefsteak plant's leaves, peppermint, Houttuynia cordata Thumb., Artemisia princeps Pump., vitamin A, vitamin B₁, B₂, B₆, B₁₂, folic acid, vitamin C, vitamin D and vitamin E.

Moreover, it would be expected that the fats and oils-containing composition according to the present invention is effective not only to the PMS symptoms, but also to the symptoms observed during the menstruation.

### EXAMPLE

The present invention will hereunder be described in more detail with reference to the following Examples, but the present invention is not restricted to these specific Examples at all.

### Example 1

To the oil derived from borage (available from Nippon Synthetic Chemical Industry Co., Ltd.), there was added 0.1 part of Lipase QL (available from Meito Sangyo Co., Ltd.) and then the resulting mixture was prepared by transesterification, with stirring, at 60° C. for 15 hours. After the completion of the transesterification reaction, the enzyme was removed through filtration, the free fatty acids present in the filtrate were removed using a thin film type evaporator, followed by the discoloration and deodorization of the filtrate to thus give a fats and oils-containing composition 1. The fats and oils-containing composition 1 was inspected for the triglyceride composition and the fatty acid composition. The results thus obtained are summarized in the following Table 1.

### Example 2

There were admixed 80 parts by mass of the oil derived from evening primrose (EPO-30 available from Tama Biochemistry Co., Ltd.) with 20 parts by mass of MCT in which caprylic acid and capric acid as the constituent fatty acids were present in a ratio, by mass, of 3/1, the resulting mixture was stirred at 120° C. under reduced pressure, followed by the deaeration and dehydration of the mixture. To the mixture, there was added 0.1 part by mass of sodium methylate and then a random transesterification reaction was performed at 120° C. for 30 minutes. The resulting reaction product was washed according to the usual method, followed by drying, discoloration and deodorization to thus give a fats and oils-containing composition 2. The fats and oils-containing composition 2 was inspected for the triglyceride composition and the fatty acid composition. The results thus obtained are listed in the following Table 1.

### Example 3

There were admixed 77 parts by mass of the oil derived from evening primrose (EPO-30 available from Tama Biochemistry Co., Ltd.) with 23 parts by mass of a middle fatty acid mixture whose ratio, by mass, of caprylic acid/capric acid was 1/1 and then 0.1 part of Lipase QL (available from Meito Sangyo Co., Ltd.) was added to the resulting mixture to thus subject the mixture to a transesterification reaction at 60° C. for 15 hours with stirring. After the completion of the transesterification reaction, the enzyme was removed through filtration, the free fatty acids present in the filtrate were removed using a thin film type evaporator, followed by the discoloration and deodorization of the filtrate to thus give a fats and oils-containing composition 3. The fats and oils-containing composition 3 was inspected for the triglyceride composition and the fatty acid composition. The results thus obtained are listed in the following Table 1.

### Example 4

To a mixture of 80 parts by mass of the oil derived from borage (available from Nippon Synthetic Chemical Industry Co., Ltd.) and 20 parts by mass of MCT in which caprylic acid and capric acid as the constituent fatty acids were present in a ratio, by mass, of 3/1, there was added 0.1 part by mass of Lipozyme (available from NOVO Industry Co., Ltd.) to thus subject the mixture to a transesterification reaction at 60° C. for 15 hours with stirring. The enzyme was removed from the reaction product through filtration and then the resulting filtrate was subjected to water washing, drying, discoloration and deodorization in this order to thus give a fats and oils-containing composition 4. The fats and oils-containing composition 4 was inspected for the triglyceride composition and the fatty acid composition. The results thus obtained are listed in the following Table 1.

### Comparative Example 1

There were admixed 80% by mass of cottonseed oil (available from The Nissin Oil Mills, Ltd.), 15% by mass of soybean oil (available from The Nissin Oil Mills, Ltd.) and 5% by mass of olive oil (available from The Nissin Oil Mills, Ltd.) to give a fats and oils-containing composition 5. The fats and oils-containing composition 5 was inspected for the triglyceride composition and the fatty acid composition. The results thus obtained are listed in the following Table 2.

### Comparative Example 2

The oil derived from borage (available from Nippon Synthetic Chemical Industry Co., Ltd.) as a fats and oils-containing composition 6 was inspected for the triglyceride composition and the fatty acid composition. The results thus obtained are listed in the following Table 2.

TABLE 1
Results of Analysis of Fats and
Oils-Containing Composition

| | Ex. No. | | | |
|---|---|---|---|---|
| | 1 | 2 | 3 | 4 |
| | Fats and Oils-Containing | | | |
| | Composition No. | | | |
| | 1 | 2 | 3 | 4 |
| γ-Linolenic acid (1-, 3-positions) | 50.2 | 50.3 | 50.4 | 41.8 |
| γ-Linolenic acid (2-position), | 49.8 | 49.7 | 49.6 | 58.2 |
| [mole %] | | | | |
| γ-Linolenic acid (1,3)/ | 1:0.99 | 1:0.99 | 1:0.98 | 1:1.39 |
| γ-Linolenic acid (2) | | | | |
| C8:0 | 0.0 | 15.0 | 17.3 | 15.5 |
| C10:0 | 0.0 | 5.0 | 5.8 | 4.5 |
| C16:0 | 10.3 | 8.2 | 5.0 | 7.6 |
| C16:1 | 0.1 | 0.1 | 0.0 | 0.1 |
| C18:0 | 4.4 | 3.5 | 2.4 | 3.7 |
| C18:1 | 17.4 | 13.9 | 7.9 | 12.8 |
| C18:2 | 36.6 | 29.3 | 37.2 | 30.4 |
| C18:3α | 0.2 | 0.2 | 0.0 | 0.2 |
| C18:3γ | 21.8 | 17.4 | 23.9 | 16.9 |
| C20:0 | 0.3 | 0.2 | 0.5 | 0.2 |
| C20:1 | 4.2 | 3.4 | 0.4 | 3.8 |
| C22:0 | 0.2 | 0.2 | 0.2 | 0.2 |
| C22:1 | 2.7 | 2.2 | 0.0 | 2.6 |
| C24:0 | 0.1 | 0.1 | 0.1 | 0.1 |
| C24:1 | 1.7 | 1.3 | 0.3 | 1.4 |
| Total [% by mass] | 100.0 | 100.0 | 100.0 | 100.0 |

TABLE 2
Results of Analysis of Fats and
Oils-Containing Composition

Comp. Ex. No.

| | |
|---|---|
| 1 | 2 |

Fats and
Oils-Containing
Composition No.

| | 5 | 6 |
|---|---|---|
| γ-Linolenic acid (1-, 3-positions) | 0 | 22.0 |
| γ-Linolenic acid (2-position), [mole %] | 0 | 78.0 |
| γ-Linolenic acid (1,3)/γ-Linolenic acid (2) | | 1:3.55 |
| C8:0 | 0.0 | 0.0 |
| C10:0 | 0.0 | 0.0 |
| C16:0 | 19.5 | 10.3 |
| C16:1 | 0.6 | 0.1 |
| C18:0 | 3.3 | 4.4 |
| C18:1 | 23.9 | 17.4 |
| C18:2 | 48.9 | 36.6 |
| C18:3α | 1.4 | 0.2 |
| C18:3γ | 0 | 21.8 |
| C20:0 | 0.4 | 0.3 |
| C20:1 | 0.1 | 4.2 |
| C22:0 | 0.3 | 0.2 |
| C22:1 | 0.0 | 2.7 |
| C24:0 | 0.1 | 0.1 |
| C24:1 | 1.5 | 1.7 |
| Total [% by mass] | 100.0 | 100.0 |

### Example 5

A feed comprising 25% by mass each of the fats and oils-containing composition 1 to 6 was freely given to 4-week-old Wistar male rats over 8 weeks. The composition of each feed is shown in the following Table 3. Soybean oil (3% by mass) was added to all of the feeds to prevent any essential fatty acid deficiency in the animals. Vitamins and minerals used herein were those recommended by the Nutrition Society in the United States and the amounts thereof added to the feeds were adjusted on the basis of the energy densities of the feeds. After 8 weeks from the initiation of the experimental feed intake, 8 animals per group were dissected to determine the mass of the visceral fat of each animal. Moreover, each dead body was freeze-dried and the content of fats thereof was determined using a Soxhlet extractor to thus evaluate the amount of the subcutaneous fat. The results thus obtained for the rats kept over 8 weeks are summarized in the following Table 4. In this test, it was confirmed that there were not observed, in every experimental groups, any statistically significant difference in the amounts of the feed intake, the final body weights and the lengths of the tails. The masses of the visceral fat tissues and the amount of the subcutaneous fat observed for the rats kept over 8 weeks showed statistically significant low values in the test groups to which the fats and oils-containing compositions 2 to 4 were administered. The results of the foregoing animal test clearly indicate that the body fat-accumulation observed for the animals, which ingested the fats and oils-containing compositions 2 to 4 having a rate of the middle chain fatty acids with respect to the total fatty acids constituting each composition falling within the range specified in the present invention, was lower than that observed for the animals to which the comparative fats and oils-containing compositions 5 and 6 were administered.

TABLE 3
Composition of Feed

| Ingredient | Amount (% by mass) |
|---|---|
| Fats and Oils-Containing | 25.0 |
| Composition | |
| Corn Starch | 25.1 |
| Casein | 25.4 |
| Sucrose | 10.0 |
| Soybean Oil | 3.0 |
| Cellulose | 5.0 |
| Mineral Mixture | 4.5 |
| Vitamin Mixture | 1.3 |
| L-Cystine | 0.38 |
| Choline Bitartrate | 0.32 |

TABLE 4
Results of Animal Test (Animals were kept over 8 weeks.)

| | Fats and Oils Composition No. | | | | | |
|---|---|---|---|---|---|---|
| | 1 | 2 | 3 | 4 | 5 | 6 |
| Amt. Of Feed Intake (g/8 | 693 ± 5 | 691 ± 6 | 698 ± 7 | 695 ± 7 | 696 ± 4 | 693 ± 5 |
| weeks) | | | | | | |
| Final Body Weight (g) | 293 ± 5 | 290 ± 5 | 288 ± 5 | 290 ± 5 | 291 ± 5 | 293 ± 5 |
| Length of Tail (cm) | 18 ± 1 | 19 ± 1 | 18 ± 1 | 18 ± 1 | 18 ± 1 | 18 ± 1 |
| Amt. Of Visceral Fat (g) | 22 ± 2 | 17 ± 1* | 18 ± 1* | 17 ± 1* | 22 ± 1 | 22 ± 2 |
| Amt. Of Subcutaneous Fat (g) | 30 ± 2 | 25 ± 2* | 25 ± 2* | 25 ± 1* | 30 ± 1 | 30 ± 2 |

Note:
The data each is expressed in terms of average value ± standard error.
*The risk percentage was found to be not more than 5% and there is observed a significant difference in the risk percentage as compared with that observed for the control group.

### Example 6

In this Example, we conducted quality stability tests and sensory tests using the fats and oils-containing compositions prepared in the foregoing Examples and Comparative Examples. More specifically, the quality stability was determined by the test for stability to oxidation according to the standard fats and oils analysis method (JOCS 2, 4, 28, 2-93 CDM Test), in which each composition was forced to undergo the self-oxidation. In addition, the taste and texture of each composition was evaluated by the sensory test. More specifically, each panelist evaluated the taste and texture of the test compositions relative to the control composition according to the following three evaluation criteria: good; identical (to the control); and bad. The results thus obtained are listed in the following Table 5.

In Table 5, the numerical values shown in this table each means the time required for arriving at each corresponding inflection point in the graph obtained by the CDM test or the forced self-oxidation test. The data listed in Table 5 indicate that the fats and oils-containing compositions 1 to 4 showed values almost two times that observed for the fats and oils-containing composition 6 (control) and this clearly indicates that the compositions of the present invention are excellent in the stability to oxidation. Contrary to this, it was found that the fats and oils-containing composition 5 has poor stability to oxidation and that it is almost identical to that observed for the control composition.

TABLE 5
Results of Quality Stability Tests and
Sensory Tests

| | | Eval. Of Taste | | |
|---|---|---|---|---|
| Fats and | Quality Stability | and Texture: | | |
| Oils-Containing | Evaluation: CDM | Sensory Test | | |
| Composition No. | Test, Inflection Point | Good | Identical | Bad |
| 1 | ◯: 8.7 | 8 | 7 | 0 |
| 2 | ◯: 7.8 | 10* | 5 | 0 |
| 3 | ◯: 8.0 | 10* | 4 | 1 |
| 4 | ◯: 8.2 | 10* | 4 | 1 |
| 5 | X: 4.0 | 6 | 9 | 0 |
| 6 (Control | 4.1 | — | — | — |
| Composition) | | | | |

Note:
The numerical value in the column entitled “Evaluation of Taste and Texture” means the number of panelists (Total number of panelists joined in this test: 15)

As a result of the independency test, it was found that the majority of the panelists statistically significantly evaluated the fats and oils-containing compositions 1 to 4 to be “good”.

(Evaluation of Quality Stability)

o: It is judged that the fats and oils-containing compositions 1 to 4 have times required for arriving at the inflection points longer than that observed for the control and that the compositions 1 to 4 are therefore quite stable.

x: The quality stability is found to be almost identical to that observed for the control fats and oils-containing composition.

### Example 7

In this Example, we conducted experiments for confirming the rate of absorbing a fats and oils-containing composition using 4-week-old Wistar male rats. The fats and oils-containing composition 6 was used as a control and the compositions 1 and 3 were used as test compositions. This test was conducted by forcing the rats to ingest a sample through the oral cavity using a catheter and the liquids were extracted, with time, from the intestinal portal vein and the lymph duct to thus analyze the fatty acids. The results thus obtained are summarized in the following Table 6.

The foregoing results suggest that the fats and oils-containing compositions 1 and 3 are transported to the liver through the portal vein and they are immediately absorbed therein in the form of free fatty acids to thus show their physiological functions. In addition, there is not any significant difference in the physiological functions between the fats and oils-containing compositions 1 and 3, but it is also suggested that there is such a tendency that the composition 3 may be excellent 10 in the immediate effect as compared with the composition 1.

TABLE 6
Changes, with Time, of the γ-Linolenic
Acid Concentration in the Fluids Extracted from Portal Vein
(P.V.) and Lymph (L)

| Fats & Oils-Containing | | | |
|---|---|---|---|
| Composition No. | Initial | After 0.5 Hr. | After 1.5 Hr. |
| 1 | P.V.: | P.V.: | P.V.: 120 |
| | 100 | 195* | L: 101 |
| | L: 100 | L: 112* | |
| 3 | P.V.: | P.V.: | P.V.: 125 |
| | 100 | 228* | L: 105 |
| | L: 100 | L: 125* | |
| 6 (Control) | P.V.: | P.V.: 112 | P.V.: 102 |
| | 100 | L: 172 | L: 131 |
| | L: 100 | | |
| Note: | | | |
| Each numerical value in the table is expressed in terms of the value relative to the γ-linolenic acid concentration (which is defined to be 100) in the | | | |
| #blood of the portal vein or the lymph at the initiation of the experiment. | | | |
| The Upper Value in Each Column: The value in the blood derived from the portal vein relative to that observed at the initiation of the experiment; The lower | | | |
| #Value in the Column: The value in the lymph relative to that observed at the initiation of the experiment. | | | |

*The risk percentage was found to be not more than 5% and there is observed a significant difference in the risk percentage as compared with that observed for the control group.

### Example 8

In this Example, we conducted tests for inspecting various fats and oils-containing compounds for the degree of PMS symptom-alleviation and the immediate effect thereof, using 98 women who suffered from the PMS symptoms.

The fats and oils-containing compositions 5 and 6 were used as control compositions. On the other hand, the fats and oils-containing compositions 1 and 3 were used as test compositions. The foregoing subjects were divided into 4 groups each comprising about 25 women (control groups and test groups) and these subjects were examined according to the blind test. The test was continued over 6 months.

Ingesta: Fats and Oils (250 mg)+Vitamin E (8 mg)/capsule

Intake and Ingestion Term: Each subject ingested each particular composition at a rate of 4 capsules/day everyday during the corpus luteum-activated phase (there is an individual difference) over 6 menstrual cycles.

Recording Method: Each subject recorded the symptoms and the degrees of seriousness thereof (according to the following 4 evaluation scores: 0 to 3) during not only the ingestion term, but also everyday (during the 2 to 3 menstrual cycles): 0=No symptom; 1=slightly anxious about the symptom; 2=anxious about the symptom; 3=the symptom interferes with the subject's life.

Evaluation Method: The records written by the individual subjects were recovered and totalized. Regarding the seriousness degrees of symptoms (evaluated using 4 scores: 0 to 3), each totalized value is expressed in terms of the numerical value obtained by determining an average of a specific subject per corpus luteum-activated phase (individual average) and then further averaging the individual averages for each test group.

The results listed in Table 7 indicate that the total scores each showing the seriousness degree per corpus luteum-activated phase of the physical symptoms and the psychic and social symptoms are statistically and significantly low even after 3 months from the initiation of the intake and that the symptoms are alleviated. This clearly indicates that there is observed a difference in effect between the compositions even if the same amount of γ-linolenic acid is ingested and that the compositions 1 and 3 show excellent symptom-mitigating effects as compared with the control composition.

In this connection, there is not any significant difference between the compositions 1 and 3, but the effect of the composition 3 may be rather higher than that 5 observed for the composition 1.

TABLE 7
Effects of Fats & Oils-Containing
Compositions for Mitigating PMS Symptoms

| | | Preliminary | After 3 | After 6 |
|---|---|---|---|---|
| Test Group | Group of Symptoms | Exam. | Months | Months |
| Test Group | Physical Symptoms | 0.72 | 0.30* | 0.20* |
| (Comp. 1) | Psychic & Social | 0.75 | 0.30* | 0.20* |
| | Symptoms | | | |
| Test Group | Physical Symptoms | 0.70 | 0.26* | 0.18* |
| (Comp. 3) | Psychic & Social | 0.74 | 0.27* | 0.15* |
| | Symptoms | | | |
| Test Group | Physical Symptoms | 0.35 | 0.32 | 0.38 |
| (Comp. 5) | Psychic & Social | 0.63 | 0.65 | 0.68 |
| | Symptoms | | | |
| Test Group | Physical Symptoms | 0.63 | 0.50 | 0.40 |
| (Comp. 6) | Psychic & Social | 0.60 | 0.70 | 0.70 |
| | Symptoms | | | |
| Note: | | | | |
| Each numerical value means the average of the total PMS score per corpus luteum-activated phase. | | | | |
| The term “Physical symptoms” means the average value of the individual evaluation concerning abdominal pains, lumbago, headaches, abdominal inflation, stiffness | | | | |
| #in the shoulders, promotion of appetite, diarrhea, constipation, mammary inflation, sleepiness and increase in discharge from the womb. | | | | |
| The term “Psychic symptoms” means the average value of the individual evaluation concerning irritation, melancholy and touchiness. | | | | |
| The term “Social symptoms” means the average value of the individual evaluation concerning feeling tired of doing anything, use of strong languages to others and desire to rearrange something and put them in order. | | | | |
| *The risk percentage was found to be not more than 5% and there is observed a significant difference in the risk percentage as compared with that observed for the | | | | |
| #control group (the fats and oils-containing composition 6 was administered). | | | | |

### Example 9

In this Example, we conducted tests for inspecting various fats and oils-containing compounds for the degree of PMS symptom-alleviation and the immediate effect thereof, using 62 women who relatively seriously suffered from the PMS symptoms. The fats and oils-containing compositions 1 and 3 were used as test compositions. The foregoing subjects were divided into 3 groups each comprising about 20 women (control groups and test groups) and capsules each containing 250 mg of fats and oils were administered to each subject in a rate of 8 capsules/day. The capsules were ingested in the corpus luteum-activated phase depending on the self-judgment of each subject. Moreover, the test was conducted according to the blind test. The test was continued over 3 months.

This Example was conducted as additional and confirmative experiments for Example 8 and therefore, the intake of the composition was increased. The test methods and evaluation methods were the same as those used in Example 8 except for the testing term and the number of capsules ingested. The results thus obtained are listed in the following Table 8. It was suggested that there is not any significant difference in the physiological functions between the fats and oils-containing compositions 1 and 3, but there is observed such a tendency that the composition 3 may be excellent in the immediate effect (efficacy) as compared with the composition 1. It would be assumed from the results shown in Table 8 that the immediate effect is not ascribed to the amount of γ-linolenic acid, but is ascribed to the difference between the amounts of γ-linolenic acid residues linked to the 1-, 3-positions and the 2-position and that the requirement for the middle chain fatty acids is likewise important in this respect.

TABLE 8
Effects of Fats & Oils-Containing
Compositions for Mitigating PMS Symptoms

| | | Initiation | |
|---|---|---|---|
| | | of | After 3 |
| Test Group | Group of Symptoms | Test | Months |
| Test Group (Fats & | Physical Symptoms | 0.83 | 0.30* |
| Oils-Containing | Psychic & Social | 0.72 | 0.30* |
| Comp. 1) | Symptoms | | |
| Test Group (Fats & | Physical Symptoms | 0.85 | 0.26* |
| Oils-Containing | Psychic & Social | 0.75 | 0.25* |
| Comp. 3) | Symptoms | | |
| Control Group (Fats | Physical Symptoms | 0.80 | 0.70 |
| & Oils-Containing | Psychic & Social | 0.80 | 0.80 |
| Comp. 6) | Symptoms | | |

Note:
Each numerical value means the average of the total PMS score per corpus luteum-activated phase.
The terms “Physical symptoms” and “Psychic and Social symptoms” are the same as those defined in connection with Table 7.
*The risk percentage was found to be not more than 5% and there is observed a significant difference in the risk percentage as compared with that observed for the control group.

### RESULTS OF EXAMPLES

The drug for mitigating the symptoms associated with the PMS, the food and drink for mitigating the symptoms associated with the PMS and the fats and oils-containing composition for mitigating the symptoms associated with the PMS, which are prepared by incorporating the glycerin fatty acid ester according to the present invention show effects of mitigating the symptoms associated with the PMS and have excellent immediate efficacy. These products can show advantageous effects even if a patient ingests the same after the outbreak of such symptoms. In addition, if the ester carries fatty acid residues having 2 to 12 carbon atoms, it is not only excellent in the absorbability, but also has a low ability of body fat-accumulation and is also excellent in the taste and texture as well as the quality stability.

Moreover, the ester can appropriately cope with every symptom and therefore, it can satisfy not only the requirements for the immediate effect, but also various consumers' needs.

### BRIEF DESCRIPTION OF THE DRAWING

FIG. 1 is a diagram illustrating typical symptoms among the premenstrual syndromes.

### DETAILED DESCRIPTION OF THE DRAWING

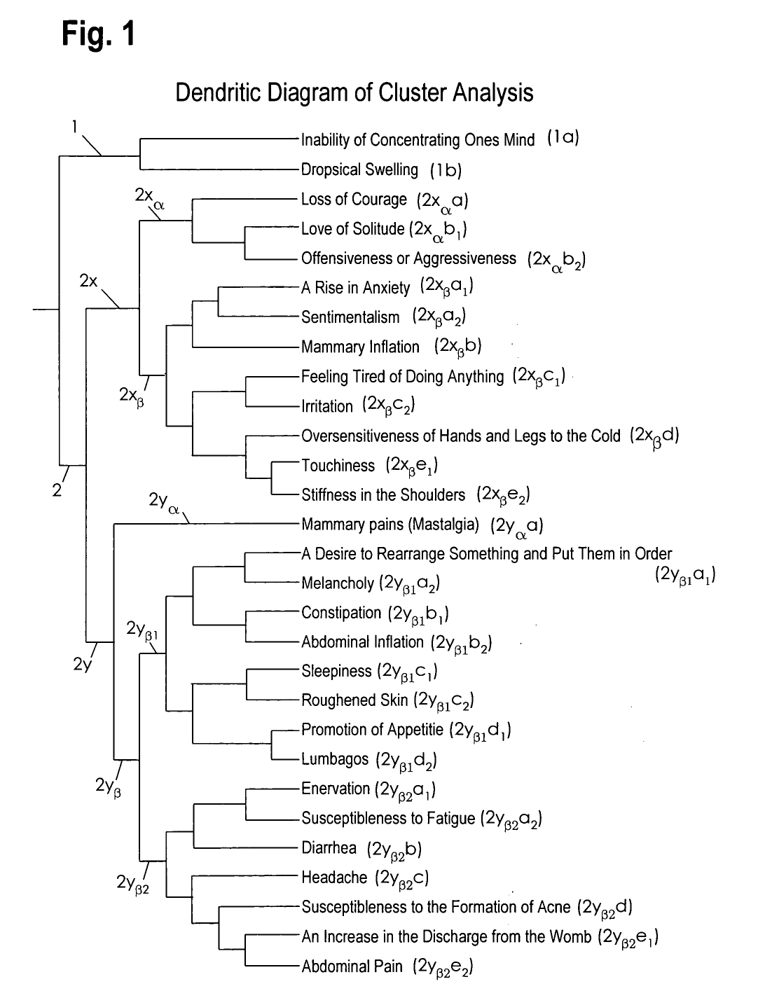

In FIG. 1 there is setforth a denddritic diagram of cluster analysis showing the following:

| 1: | 1a-Inability of Concentrating Ones Mind; | |
|---|---|---|
| | 1b-Dropsical Swelling; | |
| 2: | 2x: 2x_{α}: 2x_{α}a | Loss of Courage; |
| | 2x_{α}b₁: | Love of Solitude; |
| | 2x_{α}b₂: | Offensiveness or Aggressiveness; |
| | 2x_{β}: 2x_{β}a₁: | Rise in Anxiety; |
| | 2x_{β}a₂: | Sentimentalism; |
| | 2x_{β}b: | Mammary Inflation; |
| | 2x_{β}c₁: | Feeling Tired of Doing Anything; |
| | 2x_{β}c₁: | Irritation; |
| | 2x_{β}d: | Oversensitiveness of Hands and |
| | | Legs to the Cold; |
| | 2x_{β}e₁: | Touchiness; |
| | 2x_{β}e₂: | Stiffness in the Shoulders; |
| 2: | 2y: 2y_{α}: 2y_{α}a: | Mammary Pains (Mastalgia); |
| | 2y_{β}: 2y_{β1}: 2y_{β1}a₁: | A Desire to Rearrange Something |
| | | and Put Them in Order; |
| | 2y_{β1}a₂: | Melancholy; |
| | 2y_{β1}b₁: | Constipation; |
| | 2y_{β1}b₂: | Abdominal Inflation; |
| | 2y_{β1}c₁: | Sleepiness; |
| | 2y_{β1}c₂: | Roughened Skin; |
| | 2y_{β1}d₁: | Promotion of Appetite; |
| | 2y_{β1}d₂: | Lumbagos; |
| | 2y_{β2}: 2y_{β2}a₁: | Enervation; |
| | 2y_{β2}a₂: | Susceptibleness to Fatigue; |
| | 2y_{β2}b: | Diarrhea; |
| | 2y_{β2}c: | Headache; |
| | 2y_{β2}d: | Susceptibleness to the Formation |
| | | of Acne; |
| | 2y_{β2}e₁: | An Increase in the Discharge from |
| | | the Womb; |
| | 2y_{β2}e₂: | Abdominal pain. |

1. An edible fats and oils-containing composition for mitigating the symptoms associated with premenstrual syndromes, characterized in that it comprises a glycerin fatty acid ester for mitigating the symptoms associated with premenstrual syndromes, wherein the following substances (A) and/or (B) are prepared for use as a starting material and then subjected the substances (A) and/or (B) to transesterification, and represented by the following general formula (I):
(A): At least one member selected from the group consisting of borage oil, oils derived from evening primrose and microorganism-fermented fats and oils; and
(B): At least one member selected from the group consisting of fatty acids each having 2 to 12 carbon atoms, glycerin fatty acid esters carrying 1 to 3 fatty acid residues having 2 to 12 carbon atoms and fats and oils containing the same,
R₁O—CH₂—CH(OR₂)—CH₂—OR₃ (I)
wherein R₁, R₂ and R₃ each represents a hydrogen atom or a fatty acid residue having 2 to 24 carbon atoms, provided that at least one of R₁ and R₃ represents a γ-linolenic acid residue, at least one of R₁, R₂ and R₃ represents a fatty acid residue having 2 to 12 carbon atoms, and the total amount of the γ-linolenic acid residues present in R₁ and those present in R₃ is not less than 1% by mass on the basis of the total amount of the fatty acid residues present in the composition, and the ratio of the total amount of the γ-linolenic acid residues present in R1 and those present in R3 to that of γ-linolenic acid residues present in R₂ ranges from 1:0.1 to 1:3.

2. The edible fats and oils-containing composition for mitigating the symptoms associated with premenstrual syndromes as set forth in claim 1, wherein the total amount of the γ-linolenic acid residues present in R₁, R₂ and R₃ is not less than 2% by mass on the basis of the total amount of the fatty acid residues present in the composition.

3. The edible fats and oils-containing composition for mitigating the symptoms associated with premenstrual syndromes as set forth in claim 1, wherein the total amount of the fatty acid residues having 2 to 12 carbon atoms present in R₁, R₂ and R₃ is not less than 5% by mass on the basis of the total amount of the fatty acid residues present in the composition.

4. An edible fats and oils-containing composition for mitigating the symptoms associated with premenstrual syndromes comprising a glycerin fatty acid ester for mitigating the symptoms associated with premenstrual syndromes as set forth in claim 1, wherein the total amount of the γ-linolenic acid residues present in R₁, R₂ and R₃ is not less than 5% by mass on the basis of the total amount of the fatty acid residues present in the composition; the total amount of the fatty acid residues having 2 to 12 carbon atoms present in R₁, R₂ and R₃ is not less than 10% by mass on the basis of the total amount of the fatty acid residues present in the composition; and 40 to 90% by mass of the total amount of the γ-linolenic acid residues present in R₁, R₂ and R₃ is present in R₁ or R₃.

5. The edible fats and oils-containing composition for mitigating the symptoms associated with premenstrual syndromes as set forth in claim 1, wherein the composition is prepared by transesterifying fats and oils containing 5 to 95% by mass of γ-linolenic acid residues on the basis of the total amount of the fatty acid residues present in the composition.

6. The edible fats and oils-containing composition for mitigating the symptoms associated with premenstrual syndromes as set forth in claim 1, wherein the composition is prepared by transesterifying fats and oils containing 5 to 95% by mass of γ-linolenic acid residues and 5 to 40% by mass of fatty acid residues having 2 to 12 carbon atoms, on the basis of the total amount of the fatty acid residues present in the composition.
